(19) United States
(12) Patent Application Publication
(10) Pub. No.: US 2023/0407401 A1
(43) Pub. Date: Dec. 21, 2023

(54) DNA DAMAGE DEPENDENT MICRORNA SIGNATURE FOR CANCERS, METHODS AND USES RELATED THERETO

(71) Applicant: National Institute of Immunology, New Delhi (IN)

(72) Inventor: Sagar SENGUPTA, New Delhi (IN)

(73) Assignee: National Institute of Immunology, New Delhi (IN)

(21) Appl. No.: 17/998,888

(22) PCT Filed: Sep. 4, 2020
(86) PCT No.: PCT/IN2020/050776

§ 371 Date: Nov. 15, 2022

(30) Foreign Application Priority Data
May 29, 2020 (IN) 202011022661

(51) Int. Cl. C12Q 1/6886; C12N 15/113

(52) U.S. Cl. CPC C12Q 1/6886; C12N 15/113; C12Q 2600/178; C12Q 2600/158; C12N 2310/113

(57) ABSTRACT

The present invention related to microRNA (miR) signatures also called DNA damage sensitive miRs (DDSMs) which responds to the levels of DNA damage. The present invention provides method for identification of DDSMs which are upregulated by a common transcription factor (CDX2). Further, the present invention also provides miR inhibitors along with nanoparticle or hydrogel or adenoviral based delivery system for administering the same and kits comprising the same. The microRNA (miR) signature of the present invention can be used as biological markers to detect earliest stages of cancers particularly colon cancers. The present invention also provides a method of treatment of other types of cancer and related diseases.

### TECHNICAL FIELD

The present invention relates to the field of cell molecular biology and genetics. More specifically, the present invention relates to a microRNA (miR) signature also called DNA damage sensitive miRs (DDSMs) which responds to the levels of DNA damage. The present invention provides method for identification of DDSMs which are upregulated by a common transcription factor (CDX2). Further, the present invention also provides miR inhibitors along with nanoparticle or hydrogel or adenoviral based delivery system for administering the same and kits comprising the same. The microRNA (miR) signature of the present invention can be used as biological markers. The present invention also provides a method of treatment of cancer and related diseases.

### BACKGROUND ART

Mutations in caretaker tumour suppressor BLM helicase leads to Bloom Syndrome (BS). BS patients accumulate high levels of DNA damage leading to genomic instability and predisposition to a wide spectrum of cancers including colon cancer.

Such genomic instability causes cells to undergo neoplastic transformation. The process of neoplastic transformation, whereby the normal cells ultimately gets converted to cells with tumorigenic potential, is a key event accompanied by vast changes in gene expression profiles (Zhang, Zhou et al., 1997). For multiple types of cancers, the changes in the gene expression profiles have been shown to be associated with histopathological parameters and/or clinical outcome (Bertucci, Salas et al., 2004, van 't Veer, Dai et al., 2002). In normal cells gene expression is tightly controlled. Hence when normal cells are exposed to DNA damage, transient changes in DNA damage response and gene expression occur until the damage is repaired and homeostasis gets re-established. However, when cells have high levels of persistent endogenous DNA damage or are exposed to genotoxic stresses which can no longer be efficiently repaired, there is complete rewiring of the gene expression (Spriggs, Bushell et al., 2010), which leads to trigger mechanisms, inducible responses and genotoxic adaptations (Christmann & Kaina, 2013). If the DNA damage consistently persists as seen in tumor samples (Halazonetis, Gorgoulis et al., 2008), such rewired transcriptional programming may become a self-perpetuating feed-forward loop which ultimately has the potential to manifest as a hallmark of the disease condition itself.

The present invention provides entire workflow for the functioning of cancer specific miR signature—whereby the identity of the upstream regulator (CDX2) and the downstream effectors (DDR proteins like BRCA1, ATM, Chk1 and RNF8) have been simultaneously elucidated and validated in both mice models and patient samples. Further, this invention opens up the possibility of the usage of the DDSMs as a potential cancer prognostic biomarker, as a target for miR inhibition and usage of synthetic lethality as a treatment procedure.

### OBJECTIVES OF THE INVENTION

An objective of the present invention is to provide a microRNA (miR) signature called DDSMs which responds to the levels of DNA damage. These DDSMs are upregulated by a DNA damage inducible transcription factor i.e. CDX2.

Another objective of the invention is to provide a method for identifying changes in the micro RNA/small RNA expression profiles.

One another objective of the invention is to provide a prognostic biomarker for cancer.

An objective of the present invention is to provide miR inhibitors, a nanoparticle or hydrogel or adenoviral based delivery system and a method of treatment, diagnosis and prognosis using the same.

Another objective of the present invention is to provide a method and kit for detecting DDSMs.

### SUMMARY OF THE INVENTION

The present invention provides a microRNA (miR) signature also called DNA damage sensitive miRs (DDSMs) which responds to the levels of DNA damage. The present invention provides method for identification of DDSMs which are upregulated by a common transcription factor (CDX2). Further, the present invention also provides miR inhibitors along nanoparticle or hydrogel or adenoviral based delivery system for administering the same and kits comprising the same. The microRNA (miR) signature of the present invention can be used as biological markers to detect earliest stages of cancer. The present invention also provides a method of treatment of other types of cancer and related diseases.

### BRIEF DESCRIPTION OF DRAWINGS

The disclosure may be more fully understood by reading the following detailed description of the embodiment, with reference made to the accompanying drawings as follows:

FIG. 1: Identification and validation of BLM dependent DNA damage sensitive miRs.

A. Alteration in microRNA levels in absence or presence of BLM in GM03509 cells. Small RNA sequencing was done in isogenic cell lines derived from BS patient, GM03509 GFP-BLM Clone 4.3.4 and GM03509 GFP Clone 100. The significantly increased and decreased miRs have been indicated on either side of the trend line by dots.

B, C. Validation of miRs whose expressions were increased in absence of BLM. Two isogenic pairs of cell lines without or with BLM expression (B) GM03509 GFP-BLM Clone 4.3.4 and GM03509 GFP Clone 100 and (C) HCT116 WT and HCT116 BLM−/− were validated by western analysis with antibodies against BLM and hsp90. Indicated miRs which were upregulated in absence of BLM in (A) were validated in both the isogenic pair of cell lines by RT-qPCR analysis. Three biological replicates were done for both protein and RT-qPCR validation. Quantitation: mean±S.D. * p≤0.05, ** p≤0.001, *** p≤0.0001.

D. miRs upregulated in absence of BLM are bound to DDSM complex. HeLa S3 pREV and HeLa S3 Flag Ago2 cells were transfected with either siControl or siBLM. Lysates were made 48 hours post-transfection. These lysates were used for immunoprecipitations with either anti-GFP or anti-Flag antibody. RNA was isolated from the immunoprecipitated material and RT-qPCR was carried out to estimate the levels of the indicated miRs. Quantitation was done from three biological replicates and represented as mean±S.D. * p≤0.05, ** p≤0.001.

E, F. miRs increased in absence of BLM were DNA damage sensitive. Isogenic cell lines, GM03509 GFP-BLM Clone 4.3.4 and GM03509 GFP Clone 100 were exposed to (E) a range of HU or (F) a gradient of IR. Expression of indicated miRs were validated in both the isogenic pair of cell lines by RT-qPCR analysis under the two experimental conditions. For both experiments, the quantitation was done from three biological replicates and represented as mean±S.D. * p≤0.05.

G. Extent of DNA damage determined the levels of the damage sensitive miRs. Same as (E, F) except the experiments were carried out in the isogenic lines in asynchronous (Asyn), after HU treatment (+HU) and after post-wash (+PW) conditions. Quantitation was done from four biological replicates and represented as mean±S.D. The statistical significance was calculated relative to GM03509 GFP-BLM Clone 4.3.4 (asynchronous, Asyn). * p>0.05.

Figure S1:

A. Alteration in microRNA levels in absence or presence of BLM in GM08505 cells. Small RNA sequencing was done in isogenic cell lines derived from BS patient, GM08505 GFPBLM and GM08505 GFP. The significantly increased and decreased miRs are indicated on either side of the trend line by dots.

B, C. Validation of miRs whose expressions were increased in absence of BLM. Two isogenic pairs of cell lines without or with BLM expression (B) GM08505 GFP-BLM and GM08505 GFP and (C) SW480 siControl and SW480 siBLM were validated by western analysis with antibodies against BLM and hsp90. Indicated miRs which were upregulated in absence of BLM in (A) were validated in both the isogenic pair of cell lines by RT-qPCR analysis. Quantitation is from three biological replicates and has been represented as mean±S.D. * p≤0.05, ** p≤0.001, *** p≤0.0001.

D. Ablation of BLM in HeLa S3 pREV and HeLa S3 Flag Ago2 cells. HeLa S3 pREV and HeLa S3 Flag Ago2 cells were transfected with either siControl or siBLM. Lysates were made 48 hours post-transfection. These lysates were used for western blotting with antibodies against BLM, Flag and hsp90. Three biological replicates were carried out.

E. Extent of DNA damage determined the levels of the DDSMs. Isogenic cell lines, GM03509 GFP-BLM and GM03509 GFP were grown in Asynchronous (Asyn), +HU and +HU/PW conditions. (Left) Lysates made were probed with antibodies against BLM, RAD51, hsp90. The numbers indicated the relative quantitation of the proteins. (Middle) Immunofluorescence followed by confocal imaging done with antibodies against 53BP1 (n=100). Nuclei were stained with DAPI. Numbers indicated the percentage of cells showing the phenotype. (Right) Expression of indicated miRs were validated by RT-qPCR analysis. Quantitation was from four biological replicates and has been represented as mean±S.D. The statistical significance was calculated relative to GM03509 GFP-BLM Clone 4.3.4 (asynchronous, Asyn). * p≤0.05.

FIG. 2: DDSMs affected neoplastic transformation.

A. Inhibition of DDSMs decreased DNA damage. Comet assays were carried out in GM03509 GFP Clone 100 cells transfected with indicated miR inhibitors. (Top) Representative images of cells with comets (n=45). (Bottom) Quantitation of tail length was from three biological replicates and represented as mean±S.D. * p≤0.05.

B, C. DDSMs modulated SCEs. SCEs were carried out in (B) GM03509 GFP Clone 100 cells transfected with the indicated miR inhibitors and (C) GM03509 GFP-BLM Clone 4.3.4 cells transfected the indicated miR mimics. Quantitation was done from three biological replicates (n=40 spreads) and represented as mean±S.D. The statistical significance was calculated relative to either Inhibitor Control or Mimic Control. * p≤0.05, ** p≤0.001, *** p≤0.0001.

D, E. Inhibition of DDSMs decreased invasion and colony formation in soft agar assay. HCT116 BLM−/− cells transfected with the indicated miR inhibitors. HCT116 WT was used as a control line. (D) Matrigel invasion assay (n=6) and (E) soft agar assay colony formation assay (n=3) were carried out. (Left in both D, E) Representative images of the invasion assay and soft agar colony formation assay. (Right in both D, E) Quantitation: mean±S.D. * p≤0.05, ** p≤0.001, *** p≤0.0001.

F, I. Inhibition of DNA damage sensitive miRs decreased the rate of tumor formation in mice xenograft models. (F) HCT116 BLM−/− derived stable lines expressing GFP and the indicated miR inhibitors were injected subcutaneously into NOD SCID mice (n=6). (I) Nanoparticle encoded miRs were injected into the base of 100 cubic cm tumors generated by subcutaneously injecting HCT116 BLM−/− cells in NOD SCID mice (n=6). Days on which injection was carried out have been indicated by arrows. In both cases tumor formation was monitored over the period indicated. One representative excised tumor for each condition has been shown. Quantitation: mean±S.D. The statistical significance was calculated relative to Inhibitor Controls. * p≤0.05.

G, J. Presence of inhibitors decreased miR levels in tumors excised at the end of xenograft experiments. RNA was isolated from the tumors at the end of both xenograft models (F and I). Levels of the indicated miRs were determined by RT-qPCR analysis. The quantitation has been from RNA isolated from six mice and represented as mean±S.D. * p≤0.05, ** p≤0.001, *** p≤0.0001.

H, K. Presence of inhibitors increased BRCA1 levels in tumors excised at the end of xenograft experiments. RNA was isolated from the tumors at the end of both xenograft models (F and I). Levels of CDX2 and BRCA1 transcript were determined by RT-qPCR analysis. The quantitation has been from RNA isolated from six mice and represented as mean±S.D. * p≤0.05, ** p≤0.001.

Figure S2

A. Inhibition of DDSMs decreased the extent of DNA damage. GM03509 GFP Clone 100 cells were transfected with either Inhibitor Control or inhibitors against each of the DDSMs. Immunofluorescence followed by confocal imaging done with antibodies against 53BP1 and GFP (n=50, two biological replicates). Nuclei were stained with DAPI. Numbers indicate the percentage of cells showing the phenotype.

B-D. Characterization of HCT116 BLM−/− cells expressing DDSMs. (B) HCT116 BLM−/− cells stably expressing Inhibitor Control, Inhibitor miR-29a-5p or Inhibitor miR-96-5p were stained with antibodies against GFP. Nuclei were stained with DAPI. Two biological replicates were carried out.

(C) Levels of the respective miRs were determined in HCT116 BLM−/− cells stably expressing Inhibitor Control, Inhibitor miR-29a-5p or Inhibitor miR-96-5p by RTqPCR. Quantitation was from three biological replicates and has been represented as mean±S.D. * p≤0.05. (D) Lysates were made from HCT116 BLM−/− cells stably expressing Inhibitor

Control, Inhibitor miR 29a-5p or Inhibitor miR 96-5p. Western analysis was done with antibodies against GFP, CDX2, BRCA1, hsp90. The numbers indicated the relative quantitation of the proteins. The experiment was done two times and representative blots shown.

FIG. 3: CDX2 regulated DNA damage sensitive miRs.

A, B. CDX2 expression corelated with the extent of DNA damage in cells. RNA was isolated from the isogenic pair of cell lines HCT116 WT and HCT116 BLM−/−. Cells were grown in (A) ±HU and +PW conditions and (B) 1 hr or 6 hrs post-IR exposure. Levels of CDX2 transcript were determined by RT-qPCR analysis. The quantitation presented was from three biological replicates for both experiments and has been represented as mean±S.D. * p≤0.05.

C-E. CDX2 bound to miR promoters. Radiolabeled double stranded annealed oligos containing CDX2 binding site present in the promoters of the indicated miRs were generated. EMSAs were carried out in presence of (C) recombinant CDX2 alone in absence or presence of anti-CDX2 antibody, (D) recombinant CDX2 alone without or with increasing amounts of the cold competitor, (E) immunoprecipitated CDX2 from cells which were either left unirradiated or were exposed to IR. CDX2/DNA complexes were visualized by autoradiography. All the experiment was done three times and representative EMSAs have been presented.

F. Lack of transactivation domain of CDX2 led to decreased promoter activity of the miRs. (Left) Expression of Flag tagged CDX2 WT and CDX2 mini proteins in HCT116 WT cells were determined by western analysis with antibodies against Flag and hsp90. (Right) Luciferase based miR promoter activity were carried out with lysates expressing either CDX2 WT and CDX2 mini. Three biological replicates were done for the entire experiment. Quantitation: mean±S.D. * p≤0.05, *** p≤0.0001.

G. Mutation of the CDX2 binding site in miRs abrogated their promoter activity. Same as (F) except luciferase assays were carried out in cells expressing CDX2 WT and either the wildtype miR promoter or mutant miR promoters where CDX2 binding site had been destroyed. Quantitation was from three biological replicates and has been represented as mean±S.D. * p≤0.05, ** p≤0.001.

H. DNA binding mutants of CDX2 did not bind to miR promoters. Same as (C) except CDX2 WT or its three DNA binding mutants (CDX2 R190A, CDX2 R238A, CDX2 R242A) were used in the EMSAs. The experiment was done three times and representative EMSA has been presented.

I. DNA binding mutants of CDX2 led to decreased promoter activity of the miRs. Same as (F) except CDX2 WT or its three DNA binding mutants (CDX2 R190A, CDX2 R238A, CDX2 R242A) were expressed for western analysis and luciferase assays. Three biological replicates were done for the entire experiment. Quantitation: mean±S.D. * p≤0.05, ** p≤0.001, *** p≤0.0001.

J. DNA binding mutants of CDX2 leads to decreased miR levels. RNA was isolated from HCT116 cells expressing CDX2 WT or its three DNA binding mutants (CDX2 R190A, CDX2 R238A, CDX2 R242A). Levels of the indicated miRs were determined by RT-qPCR analysis. Quantitation was from three biological replicates and has been represented as mean±S.D. The statistical significance was calculated relative to CDX2 WT. * p≤0.05, **≤0.001, ***≤0.0001.

K. Ablation of CDX2 led to decreased miR levels. HCT116 WT or HCT116 BLM−/− cells were transfected with either siControl or siCDX2. (Left) Levels of CDX2 were determined by western analysis with antibodies against CDX2 and hsp90. The numbers indicated the relative quantitation of the proteins. (Right) RNA isolated from both cell types transfected with siControl or siCDX2. Levels of the indicated primary, precursor or mature miRs were determined by RT-qPCR analysis. Quantitation of RT-PCR was from three biological replicates and has been represented as mean±S.D. The statistical significance was calculated relative to HCT116 WT siControl. * p≤0.05, ** p≤0.001, *** p≤0.0001.

Figure S3

A, B. Absence of BLM enhanced the level of CDX2. Isogenic cell lines, GM03509 GFP-BLM Clone 4.3.4 and GM03509 GFP Clone 100 were exposed to (A) a range of HU or (B) a gradient of IR. Expression of CDX2 was validated in both the isogenic pair of cell lines by RT-qPCR analysis under the two experimental conditions. For both experiments, quantitation was from three biological replicates and has been represented as mean±S.D. * p≤0.05.

C. Recombinant CDX2 bound specifically to its recognition sequence on miR promoters. Radiolabelled double stranded annealed oligos containing either CDX2 binding site (WT) or where CDX2 binding site was destroyed (MT) were generated. EMSAs were carried out in presence of recombinant CDX2. CDX2/DNA complexes were visualized by autoradiography. The experiment was done three times and representative EMSAs are presented.

D. Lack of transactivation domain of CDX2 led to decreased promoter activity of the miRs. Luciferase based miR promoter activity were carried out with lysates expressing either CDX2 WT or CDX2 mini. Quantitation was from three biological replicates and has been represented as mean±S.D. * p≤0.05.

E. Ablation of CDX2 led to decreased levels of mature DDSMs. HCT116 WT or HCT116 BLM−/− cells were transfected with either siControl or siCDX2. RNA isolated from each of the cell types was quantitated by RT-qPCR analysis for the levels of the indicated mature miRs.

Quantitation was from three biological replicates and has been represented as mean±S.D.* p≤0.05.

F. BRCA1 transcript levels increased when CDX2 could not bind to DNA. HCT116 cells were transfected with either CDX2 WT or the CDX2 mutants (R190A, R238A, R242A). CDX2 and BRCA1 transcript levels were determined by RT-qPCR. Quantitation was from three biological replicates and has been represented as mean±S.D. The statistical significance was calculated relative to CDX2 WT. * p≤0.05.

FIG. 4: CDX2 regulated miR dependent in vivo dissemination of colon cancer cells.

A. CDX2 was induced in TW6 cells. Lysates were made from TG8 and TW6 cells grown in ±HU conditions, in presence of Doxycycline (Dox). Western blots were carried out with antibodies against CDX2, GFP, BRCA1 and hsp90. The numbers indicated the relative quantitation of the proteins. Three biological replicates were done.

B. DDSMs were induced by CDX2 expression. RNA was isolated from TG8, TW6 cells grown in ±HU conditions and in presence of Dox. Levels of the indicated miRs were determined by RT-qPCR analysis. Quantitation was from four biological replicates and has been represented as mean±S.D. The statistical significance is calculated relative to TG8 (asynchronous, Asyn). * p≤0.05.

C, D. Induction of CDX2 led to enhanced wound healing and colony formation. (C) Scratch assay (n=3) and (D) Colony formation assay (n=3) were carried out with TG8, TW6 in ±Dox conditions. The time allowed (in hrs) for wound healing has been indicated. (Left in both C, D) Representative images of the wound healing and clonogenic assay. (Right in both C, D) Quantitation: mean±S.D. * p≤0.05.

E. Induction of CDX2 led to enhanced tumor formation in mice xenograft model. Tumor formation in a mice xenograft model was carried out by injecting TG8 and TW6 cells subcutaneously into NOD SCID mice. Mice were fed with Dox every day. Tumor formation was monitored over the period indicated. Six mice were used for each condition. One representative tumor for each condition has been also represented. Quantitation: mean±S.D. The statistical significance was calculated by comparing +Dox condition with −Dox condition. ** p≤0.001, *** p≤0.0001.

F-H. Induction of DDSMs, proliferation and angiogenesis markers in CDX2 induced tumors derived in a xenograft model. (F) RNA and (G) protein were extracted from the tumors at the end point of the xenograft experiment (E). (F) Levels of the indicated miRs were determined by RT-qPCR analysis (from 6 mice) while (G) protein levels of CDX2, GFP, PCNA, BRCA1, hsp90 in the tumors was determined by carrying out western analysis with the indicated antibodies (from 4 mice). The numbers indicated the relative quantitation of the proteins. (H) IHC was carried out with tumor sections with anti-CD31 antibodies (from 4 mice). (Left) Representative images of CD31 staining. (Right) Quantitation: mean±S.D. * p≤0.05.

I-K. Induction of CDX2 dependent miR expression led to increase in vivo dissemination of cancer cells. In vivo dissemination of GFP expressing TG8 and TW6 cells were determined in (I) sub-cutaneous model, (J) intravenous model and (K) orthotopic model. Cells were appropriately injected/implanted into mice and the mice were fed with Dox every third day. At the end of 21 days in vivo imaging of the mice (both ventral and dorsal) were carried out. Five mice were used for each condition in all the three models. (Left) Representative images of TG8, TW6 cell migration has been presented. (Right) Quantitation: mean±S.D. * p≤0.05.

Figure S4

A. Flag tagged CDX2 was overexpressed in HW2 cells. Lysates were made from HC1 and HW2 cells grown in ±HU conditions. Western blots were carried out with antibodies against Flag, CDX2 and hsp90. The experiment was done three times and representative blots shown.

B. DDSMs were induced by CDX2 expression in HW2 cells. RNA was isolated from HC1, HW2 cells grown in ±HU conditions. Levels of the indicated miRs were determined by RTqPCR analysis. Quantitation was from four biological replicates and has been represented as mean±S.D. The statistical significance was calculated relative to HC1 (asynchronous, Asyn) condition. * p≤0.05.

C. Overexpression of CDX2 led to enhanced tumour formation in mice xenograft model. Tumor formation in a mice xenograft model was carried out by injecting HC1 and HW2 cells subcutaneously into NOD SCID mice. Tumour formation was monitored over the period indicated. Six mice were used for each condition. One representative tumour for each condition has been represented. Quantitation: mean±S.D. * p≤0.05.

D-F. Induction of DDSMs, proliferation and angiogenesis markers in CDX2 overexpressing tumors derived in a xenograft model. (D) RNA and (E) protein were extracted from the tumors at the end point of the xenograft experiment (C). (D) Levels of the indicated miRs were determined by RT-qPCR analysis (from 6 mice). Quantitation: mean±S.D. * p≤0.05. (E) Levels of CDX2, GFP, PCNA, BRCA1, hsp90 protein levels in the tumors were determined by carrying out western analysis with the corresponding antibodies (from 3 mice). The numbers indicated the relative quantitation of the proteins. (F) IHC was carried out with tumor sections with anti-CD31 antibodies (from 3 mice). (Left) Representative images of CD31 staining. (Right) Quantitation of IHC staining was represented by mean±S.D. * p≤0.05.

FIG. 5: BRCA1 was identified as a target of the DDSMs.

A-D. Ablation of miRs enhanced the transcript levels of its targets. GM03509 GFP Clone 100 cells were transfected with the indicated miR inhibitors. The transcript levels of (A) BRCA1, (B) ATM, (C) Chk1, (D) RNF8 were determined by RT-qPCR. Quantitation was from three biological replicates and has been represented as mean±S.D. The statistical significance was calculated relative to Inhibitor Control. * p≤0.05, ** p≤0.001.

E-H. Overexpression of miRs enhanced the transcript levels of its targets. GM03509 GFP-BLM Clone 4.3.4 cells were transfected with miR mimics. The levels of (E) BRCA1, (F) ATM, (G) Chk1, (H) RNF8 were determined by RT-qPCR. Quantitation was from three biological replicates and has been represented as mean±S.D. The statistical significance was calculated relative to Mimic Control* p≤0.05, ** p≤0.001.

I, J. Levels of miRs determined the protein levels of miR targets. (I) GM03509 GFP Clone 100 cells or (J) GM03509 GFP-BLM Clone 4.3.4 cells were transfected with either (I) miR inhibitors or (J) miR mimics. Levels of ATM, Chk1, RNF8, BRCA1, hsp90 were determined by carrying out western analysis with the corresponding antibodies. The numbers indicated the relative quantitation of the proteins. Three biological replicates were done for both experiments.

K. DDSMs did not bind to the mutated 3′UTR of BRCA1. Luciferase assays were carried out with extracts from HCT116 cells transfected with either mimic Control or the mimics of the DDSMs being tested in presence of either the BRCA1 3′UTR WT or BRCA1 3′UTR MT. Quantitation was from three biological replicates and has been represented as mean±S.D. * p≤0.05.

Figure S5:

A. Common targets of DDSMs. Using miRanda the targets of each of the DDSMs were determined. The Venn Diagram showed the common targets of the DDSMs.

B. Transcript levels of BRCA1 were enhanced upon inhibition of the miRs. HCT116 BLM−/− cells were transfected with either inhibitor Control or specific miR inhibitors. Transcript levels of BRCA1 were determined by RT-qPCR. Quantitation was from three biological replicates and has been represented as mean±S.D. The statistical significance was calculated relative to Inhibitor Control. * p≤0.05.

C. Transcript levels of BRCA1 were decreased upon overexpression of the miRs. HCT116 WT cells were transfected with either mimic Control or specific miR mimics. Transcript levels of BRCA1 were determined by RT-qPCR. Quantitation was from three biological replicates and has been represented as mean±S.D. The statistical significance was calculated relative to Mimic Control. * p≤0.05, ** p≤0.001.

D. Protein levels of BRCA1 were enhanced upon inhibition of the miRs. Same as (B) except protein levels of BRCA1 were determined. Western blots were carried out with antibodies against BRCA1 and hsp90. The numbers indicated the relative quantitation of the proteins. The experiment was done two times and representative blots shown.

E. Protein levels of BRCA1 were decreased upon overexpression of the miRs. Same as (D) except mimic Control or specific miR mimics were used to transfect the cells. The experiment was done two times and representative blots shown.

F, G. BRCA1 transcript levels were decreased in cells exposed to different types and amounts of DNA damage. BRCA1 transcript levels in GM03509 GFP-BLM Clone 4.3.4 and GM03509 GFP Clone 100 cells exposed to (F) a gradient of HU or (G) different doses of IR, were determined by RT-qPCR. Quantitation was from three biological replicates and has been represented as mean±S.D. * p≤0.05.

H, I. BRCA1 transcript levels were decreased in cells with high levels of CDX2. Same as (F) except RT-qPCR were carried out to determine BRCA1 transcript levels in (H) TG8, TW6 cells (both in +Dox condition), (I) HC1, HW2 cells. For both experiments, quantitation was from three biological replicates and has been represented as mean±S.D. * p≤0.05.

J. Binding sites for DDSMs were present in BRCA1 3′UTR. Sequence analysis was carried out to determine the alignment of the BRCA1 3′UTR sequences with the seed sequences of the DDSMs.

FIG. 6: BLM repressed CDX2 transcription.

A. CDX2 protein levels increased in absence of BLM. Lysates made from HCT116 WT and HCT116 BLM−/− cells were probed with antibodies against CDX2 and hsp90. The numbers indicated the relative quantitation of the proteins. The experiment was done three times and representative blots presented.

B. Expression of BLM decreased CDX2 transcript levels. HCT116 BLM−/− cells were transfected with either EGFP or EGFP-BLM. BLM (left) and CDX2 (right) transcript levels were determined by RT-qPCR. Quantitation is from four biological replicates and has been represented as mean±S.D. * p≤0.05.

C. BLM was recruited to the CDX2 promoter. BLM ChIP were carried out with chromatin isolated from GM03509 GFP-BLM Clone 4.3.4 and GM03509 GFP Clone 100 cells. The recruitment of BLM to the putative binding sites of the transcriptional repressors and activator has been shown. The corresponding IgG was used as the antibody control. As specificity control, the recruitment of BLM to the GAPDH promoter was also determined. Quantitation was from four biological replicates and has been represented as mean±S.D. * p≤0.05, ** p≤0.001.

D, E. Transcriptional repressors were recruited to the CDX2 promoter. Same as (C) except ChIP was carried out with antibodies against (D) SMAD3, (E) AP2□. Quantitation was from three biological replicates and has been represented as mean±S.D. * p≤0.05.

F, G. Co-repressor complexes were recruited to the CDX2 promoter. Same as (C) except ChIP was carried out with antibodies against (F) Sin3b (G) CHD4. Quantitation was from four biological replicates and has been represented as mean±S.D. * p≤0.05.

H, I. Histone deacetylases were recruited to the CDX2 promoter. Same as (C) except ChIP was carried out with antibodies against (H) HDAC1 (I) HDAC2. HDAC1 ChIP was done three times and HDAC2 ChIP was done four times. Quantitation: mean±S.D. * p≤0.05, ** p≤0.001.

J, L. Ablation of Sin3b and CHD4 enhanced CDX2 transcript. HCT116 cells were transfected with either (J) shScramble and shSin3b or (L) siControl and siCHD4. CDX2 transcript levels were determined by RT-qPCR. Quantitation was from three biological replicates and has been represented as mean±S.D. * p≤0.05.

K, M, N, O. Ablation of transcriptional co-repressor complexes enhanced CDX2 protein levels. HCT116 cells were transfected with either (K) shScramble and shSin3b; (M) siControl and siCHD4; (N) siControl and siHDAC1 and (O) siControl and siHDAC2. Western blots were carried out with the indicated antibodies. The numbers indicated the relative quantitation of the proteins. Each of the experiments was carried out for three biological replicates, representative blots has been shown.

P. BLM was recruited to the CDX2 promoter in the adjacent normal samples of colon cancer patients from India. ChIP with anti-BLM antibody was carried out on colon cancer samples and their adjacent normal control (n =8). The amount of BLM recruitment to the different transcriptional repressor sites were determined, quantitated and represented in form of a heat-map. Three technical replicates were done for each ChIP and the mean value taken for plotting the heat map.

Figure S6:

A, B. Depletion of BRCA1 levels. GM03509 GFP BLM Clone 4.3.4, HCT116 or TG8 cells were either transfected with siControl or siBRCA1. The levels of BRCA1 were determined at the (A) RNA level by RT-qPCR or (B) protein levels by western analysis with antibodies against BRCA1 and hsp90. Quantitation was from three biological replicates and has been represented as mean±S.D. * p≤0.05.

C-E. Ablation of BRCA1 increased the amount DNA damage, SCEs and invasion. Cells obtained from (A, B) were subjected to (C) Comet assays (n=40) (D) SCEs (n=30 metaphase spreads) and (E) invasion assays (n=6). Quantitation: mean±S.D. * p≤0.05, *** p≤0.0001.

F. Overexpression of BRCA1 levels. HCT116 BLM−/− cells were transfected with HA-BRCA1 or Vector. The lysates were probed with antibodies against HA and hsp90. The experiment was done twice, and representative blots presented.

G-1. Overexpression of BRCA1 decreases the amount DNA damage, SCEs and invasion. Cells obtained from (F) were subjected to (G) Comet assays (n=40) (H) SCEs (n=25 metaphase spreads) and (I) invasion assays (n=6). Quantitation: mean±S.D. * p≤0.05, ***p≤0.0001.

FIG. 7: Levels of DDSMs are increased in colon cancer patients

A, B. Increased levels of DDSMs were observed in the tissues and blood samples of colon cancer patients in TCGA database. The expression levels of the DDSMs was quantitated in (A) tissues (B) blood of the four stages of colon cancer patients and normal individuals available in TCGA database. Quantitation: median±range. * p≤0.05, ** p≤0.005, *** p≤0.0005, **** p≤0.00005.

C, D. Increased levels of DDSMs was observed in the tissues and blood samples of colon cancer patients from India. The levels of the DDSMs were analyzed by RT-qPCR in (C) colon cancer tissues and their adjacent normal tissues in the Indian cohort, (D) blood from colon cancer patients in the Indian cohort and healthy normal individuals. Patients in stages I/II and III/IV have been combined together. Quantitation: median±range. * p≤0.05, ***≤0.0001.

E, F. Survival function of colon cancer patients increased with decreased expression of the six DDSM signature. Keplan-Meier curves were generated to determine the survival function of the colon cancer patients showing the combined expression of six miR signature in (E) colon cancer patient tissues and (F) blood from colon cancer patients. p value has been indicated.

G. Expression of the six DDSM signature inversely correlated with BRCA1 expression. Spearman correlation analysis was carried out for the expression levels of six DDSM signature and BRCA1. The correlation coefficient R and p value have been indicated.

Figure S7:

A. CDX2 levels increased in absence of BLM. Immunofluorescence was carried out with HCT116 WT and HCT116 BLM−/− cells (n=200, two biological replicates). Staining was done with antibodies against CDX2. DNA is stained with DAPI. Quantitation was from both biological replicates and is represented as mean±S.D. * p≤0.05.

B. Schematic diagram of CDX2 promoter. Approximately 5kb upstream to the TSS in CDX2 promoter was analysed. Binding sites for the different transcriptional repressors have been indicated.

C-G. BLM interacted in vivo with components of the Sin3b and NuRD co-repressor complexes and SMAD3. HCT116 cells were transfected with the indicated plasmids. Reciprocal immunoprecipitations were carried out with antibodies against (C-F) BLM or the corresponding IgG or (G) Sin3b or the corresponding IgG. The immunoprecipitates were probed with antibodies against (C) BLM, Flag, Sin3b, HDAC1, SMAD3, (D) BLM, CHD4, HDAC1, (E) BLM, Flag, HDAC1, (F) BLM, Flag, SMAD3, (G) Sin3b, GFP. The experiment was done three times and representative blots shown.

H. BLM directly interacted with SMAD3 and HDAC1. (Top) In vitro interactions were carried out with S35 methionine radiolabelled BLM and bound (left) GST and GST SMAD3, (right) GST and GST HDAC1. Post-interaction the bound radioactive BLM was detected by autoradiography. (Bottom) Inputs used for in vitro translated BLM (detected by autoradiography) and GST, GST SMAD3, GST HDAC1 (detected by Coomassie). The

experiment was done three times and representative blots shown.

I, J. BLM was bound with Sin3b and CHD4 on the CDX2 promoter. Sequential re-ChIP assays were carried out with (I) Sin3b, BLM and (J) CHD4, BLM combinations. Three indicated binding sites of the transcriptional repressors were chosen to check for the recruitment. The corresponding IgGs were used as the antibody controls. As specificity control, the recruitment of BLM to the GAPDH promoter was also determined. Quantitation was from three biological replicates and has been represented as mean±S.D. **≤0.001.

FIG. 8: Schematic diagram showing the upregulation of DDSMs in colon cancer cells.

Colon cancer cells have higher levels of damaged DNA compared to the surrounding normal cells. DNA damage led to upregulation of CDX2, which allowed CDX2 to bind to the promoters of DDSMs. The levels of DDSMs increased which caused a decrease in the levels of its targets involved in DNA damage response and DNA repair (like BRCA1). In normal cells, CDX2 expression was transcriptionally repressed as BLM recruited co-repressor complexes (Sin3b and NuRD) to the CDX2 promoter. Lack of CDX2 induction prevented the upregulation of DDSMs, due to which the level of BRCA1 remained elevated.

Figure S8:

A, B. Levels of BRCA1 mRNA decreased in the cancerous tissues and serum of colon cancer patients from India. RNA isolated from (A) colon cancer tissues and their adjacent normal tissues in the Indian cohort, (B) blood from colon cancer patients in the Indian cohort and healthy normal individuals. Quantitation: median±range. * p≤0.05, ** p≤0.001.

C, D. Levels of BRCA1 protein decreased in the colon cancer tissues in the Indian cohort as detected by Western blot analysis. (C) Western analysis of the tissue extracts from representative twelve pairs of Indian colon cancer patients (designated as C) and their adjacent normal tissues (designated as N) were carried out with antibodies against BRCA1, hsp90. (D) Quantitation of protein levels of western analysis: median±range. ** p≤0.001, *** p≤0.001.

E, F. Levels of BRCA1 protein decreased in the colon cancer tissues in the Indian cohort as detected by immunohistochemistry. (E) BRCA1 staining was detected using immunohistochemistry in two representative colon cancer tissues and their adjacent normal

tissues in the Indian cohort. (F) Quantitation of protein levels by IHC: median±range. * p≤0.05, ** p≤0.001.

### DETAILED DESCRIPTION

At the very outset, it may be understood that the ensuing description only illustrates a particular form of this invention. However, such a particular form is only an exemplary embodiment, without intending to imply any limitation on the scope of this invention. Accordingly, the description is to be understood as an exemplary embodiment and teaching of invention and not intended to be taken restrictively.

Throughout the description, the phrases “comprise” and “contain” and variations of them mean “including but not limited to”, and are not intended to exclude other moieties, additives, components, integers or steps. Thus, the singular encompasses the plural unless the context otherwise requires. Wherever there is an indefinite article used, the specification is to be understood as contemplating plurality as well as singularity, unless the context requires otherwise.

Features, integers, characteristics, compounds, chemical/biological moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification including any accompanying claims, abstract and drawings or any parts thereof, or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference. Post filing patents, original peer reviewed research paper shall be published.

The following descriptions of particular embodiments and examples are offered by way of illustration and not by way of limitation.

Unless contraindicated or noted otherwise, throughout this specification, the terms “a” and “an” mean one or more, and the term “or” means and/or.

The present invention is related to the identification of DNA damage sensitive miRs (DDSMs) which are upregulated by a common transcription factor (CDX2). The inventors have found that these miRs are not induced in cells lacking DNA damage because CDX2 promoter is epigenetically silenced via BLM-dependent recruitment of HDAC1/2 containing Sin3b and NuRD complexes. These DNA damage sensitive miRs target multiple key proteins involved in DNA damage sensing and repair (like BRCA1, ATM, Chk1, RNF8), downregulate their expression and thereby allow neoplastic transformation to occur. The enhanced expression of six of these miRs occur in different stages of colon adenocarcinoma tissues and their blood samples. Further, Kaplan-Meier analysis revealed that higher expression of the six miR signature led to lesser survival probability. Hence, this invention serves as an integrated study where inventors have demonstrated how a miR signature, normally epigenetically silenced, becomes deregulated during DNA damage, thereby represses genome stabilizers and subsequently promotes oncogenesis.

In a principal embodiment, the present invention provides a Deoxyribo-Nucleic Acid (DNA) damage sensitive microRNA signatures (DDSMs) which respond to level of DNA damage, having the sequence selected from SEQ ID NO. 1, 2, 3, 4, 5, 6, 7, 8, 9, 10, 11, 12, 13, 14, 15 and 16.

In yet another embodiment, said DDSMs are upregulated by a DNA damage inducible transcription factor.

In still another embodiment, said DNA damage inducible transcription factor is CDX2.

In another embodiment, said DDSMs find application as prognostic and diagnostic biomarkers.

In still another embodiment, said DDSMs are for qualitative and quantitative estimation of specific microRNA levels in different stages in colon cancer patients.

In yet another embodiment, said DDSMs are for the detection of colon cancer.

In still another embodiment, said detection is performed in sample selected from tissue, body fluids wherein the body fluids are blood, plasma, urine, sputum etc.

In another embodiment, the present invention provides a process of diagnosing tumor growth by detecting DDSMs, where said DDSMs are upregulated by CDX2.

In still another embodiment, said DDSMS when upregulated decrease the expression of DNA damage protein selected from BRCA1, ATM, RNF8 or Chk1.

In another important embodiment, the present invention provides a method for identification of DDSMs, wherein said method comprises the steps of:
a. isolating RNA from two pair of isogenic cell lines with or without BLM helicase;
b. conducting small RNA sequencing with isolated RNA of step (a);
c. observing expression levels of micro RNAs (miRNAs, miRs) in both the above isogenic pairs in absence of BLM expression;
d. validating relative expression of upregulated miRs obtained from step (b) in the same isogenic pairs of cells;
e. further validating the expression of upregulated miRs in isogenic lines of colon cancer origin;
f. identifying the DDSMs having SEQ IDs no. 1-16 which are upregulated by common transcription factor CDX2.

In still another embodiment, said isogenic pairs of cells are selected from immortalized cells from GM03509 complemented with either GFP-BLM (Clone 4.3.4) or GFP (Clone 100) or immortalized cells from another BS patient GM08505 complemented with either GFP-BLM or GFP.

In yet another embodiment, said method comprises administering miR inhibitors into the tumours of the colon cancer patients against the DDSMs selected from SEQ ID NO. 1, 2, 3, 4, 5, 6, 7, 8, 9, 10, 11, 12, 13, 14, 15 and 16.

In still another embodiment, said miR inhibitors are delivered along with nanoparticle or hydrogel or adenoviral based delivery system.

In another important embodiment, the present invention provides a kit for detecting DDSMs, wherein said kit comprises a microfluidic system in which the patient body fluid is added and RNA extracted is converted into complementary DNA (cDNA) by reverse transcription PCR (RT-PCR) and the level of cDNA quantitatively determined.

Among the many factors which control gene expression, one the most important is the expression of the microRNAs (miRs). The functions of miRs on genome instability and ultimate progression to different types of cancers have been well studied. It is interesting to note that changes in the miR expression levels occur during all the above processes, by generating a threshold in target gene expression (Mukherji, Ebert et al., 2011). Microarray expression or small RNA sequencing data from a large number of different cancers show both increased or decreased miR levels (Chung, Chang et al., 2017, Croce, 2009). A significant number of miRs have been reported to be dysregulated in multiple types of cancers including colorectal carcinoma (CRC). For CRC the networks governing miR-mRNA interactions have been discovered. Novel miRNA signatures have been identified and some of them validated in patient tissues (Chen, Xia et al., 2019, Ding, Lan et al., 2018). Extracellular miRNA markers from blood and faecal samples from CRC patients have also been reported, a few of which have been claimed to be diagnostic markers for the grades and pathologic stages of the disease progression (Chen et al., 2019).

To prevent the development of cancers, maintenance of genomic integrity is of utmost importance. DNA damage repair (DDR) is a default response of normal cells when exposed to multiple types of DNA damages like stalled replication, ionizing irradiation (IR), ultraviolet lights (UV) and genotoxic drugs (Ciccia & Elledge, 2010, Tikoo & Sengupta, 2010). Different types of factors regulate this response. One of the factors regulating genome integrity is BLM helicase. BLM helicase plays a role in both sensing and repairing of multiple types of damage including stalled replication and formation of double strand breaks (Sengupta, Robles et al., 2004, Tikoo, Madhavan et al., 2013, Tripathi, Agarwal et al., 2018). MRN and ATM are upstream factors which recognize and accumulate at the double strand breaks within seconds after the IR exposure (Smith, Tho et al., 2010, Syed & Tainer, 2018). Multiple other factors take part in this choreographed process including E3 ligase like RNF8 (which facilitate recruitment of DNA damage response proteins) (Zhou, Yi et al., 2019), damage specific kinases like Chk1 and Chk2 (Smith et al., 2010) and BRCA1. Tumor suppressor BRCA1 maintains genome stability by being a part of multiple protein complexes and is involved in DNA damage repair, DNA damage-induced cell cycle checkpoint activation, protein ubiquitination, transcriptional regulation and apoptosis (Savage & Harkin, 2015). Loss of any of the genome stabilizers (like BLM, ATM, BRCA1) led to accumulation of DNA damage which progresses to neoplastic transformation, cancer predisposition and finally the development of cancer (Hanahan & Weinberg, 2011, Negrini, Gorgoulis et al., 2010).

To determine whether in human cancer cells, a miR dependent unidirectional feedforward loop existed which responded to the high levels of the intracellular DNA damage and thereby caused the upregulation of a miR signature, the present inventors identified such DNA damage dependent miR signature and analyzed it relation with neoplastic transformation.

The present invention the inventors provides identification of a miR signature (called DDSMs) which can respond to the levels of intracellular DNA damage (FIG. 8). The DDSMs were transcriptionally upregulated by a common transcription factor, CDX2 (FIG. 3C-3K, S3C-S3F). CDX2 is highly expressed in colorectal carcinoma (Moskaluk, Zhang et al., 2003) and itself was upregulated after DNA damage (FIG. 3A, 3B, S3A, S3B). While CDX2 inducible miRs targeted several key proteins involved in the maintenance of genomic integrity (BRCA1, ATM, Chk1 and RNF8) (FIG. 5, S5), its own expression under normal conditions was kept in a repressed state by BLM which recruited HDAC1/HDAC2 containing co-repressor complexes, Sin3b and NuRD, to its promoter (FIG. 6, S7). The inventors also provided evidence that the ablation and upregulation of the DDSMs reciprocally affected tumor growth and progression in multiple in vivo mice models (FIG. 2F, 2I, 4E, 4I-4K, S4C). Finally, the levels of DDSMs were upregulated in cancerous tissues and blood, as analyzed from two independent cohorts of colon cancer patients (FIG. 7A-7D) and is associated with lower overall survival (FIG. 7E, 7F). It is known that most miRs are repressed in cancer relative to their normal tissues (Lu, Getz et al., 2005), a fact also supported by enhanced cell transformation and tumorigenesis in vivo when miR processing machinery is depleted (Kumar, Pester et al., 2009, Lambertz, Nittner et al., 2010). Hence the fact that DDSMs are upregulated, responds to the extent of DNA damage and has proliferative properties classifies them as a DNA damage specific oncogenic miR signature.

There are different kinds of miRs signatures that can predict CRC. These include: (a) a five-miR signature which was identified through bioinformatics and subsequently validated in the tissues from two cohorts of patients (Ozawa, Kandimalla et al., 2018); (b) an eight miR signature identified by three independent miR expression profile analysis which predicts recurrence of tumors in stages II and III CRC patients (Kandimalla, Gao et al., 2018); (c) a four miR signature which predicts relapse after curative surgery (Grassi, Perilli et al., 2018); (d) a three miR signature which predicts both distant metastasis and hepatic recurrence (Coebergh van den Braak, Sieuwerts et al., 2018) and (e) a 16-miR signature which serves as a prognostic biomarker for Stage II and III CRC patients (Jacob, Stanisavljevic et al., 2017). Distilling the results from these studies indicated that there was hardly any overlap in the list of miRs which are predicted to be upregulated in different cohorts. Indeed, apart from miR-182, none of the DDSMs overlap with any of the above studies. Such diverse results are possibly due to the fact that multiple miRs can regulate the same pathway. Such redundancy can result in cooperative functioning between the miRs where the miR functions maybe temporal and/or spacial — thereby switching on and off and finely modulating the expression levels of the target genes. Hence, it is quite possible some or maybe even all the above signatures are true and just reflect the diverse biological processes which characterize neoplastic transformation leading to the manifestation of colon cancer. Multiple studies have also predicted upregulated miRs in the sera of CRC patients which can serve as a prognostic marker (Liu, Zhou et al., 2013, Tsukamoto, Iinuma et al., 2017, Zhu, Huang et al., 2017). Interestingly, the present inventors found that the DDSMs which were upregulated in tissues were also upregulated in the blood of the patients (FIG. 7A-7D). This result indicated that the DDSMs containing tumor cells are present in the blood of the patients and/or are actively secreted from the colorectal cancer cells, possibly reflecting their high levels within the colon cancer tissues. The strong correlation observed for lower expression of the DDSMs with better overall survival (FIG. 7E, 7F), indicates how the six-miR signature maybe used as a prognostic marker for colon cancer progression.

The wound healing and xenograft experiments done in the present invention (FIG. 4, S4) are in contrast to the ones reported earlier on colon cancer cells reported by the inventors (Gross, Duluc et al., 2008) and others (Mallo, Soubeyran et al., 1998, Yu, Liu et al., 2019). Further, it has also been reported that CDX2 is a key functional regulator of the molecular (extracellular matrix molecules, cytokines) and cellular (T cell repertoire) environment in tumours (Balbinot, Armant et al., 2018). Much of the difference is due to the difference in the experimental protocols employed in this report. Specifically, for the xenograft experiments were done very different strains (NOD SCID vs nude mice) and in the present invention the cells were embedded into matrigel. The use of matrigel, on one hand and the strongly immune-deficient NOD SCID mice compared to nude mice, on the other hand, put the cancer cells in a very different microenvironment compared to the experiments which were earlier performed. Indeed, the results of the present invention which point towards the oncogenic potential of CDX2 overexpression. For example, amplification of CDX2 genomic loci is observed in colon cancer and is essential for the proliferation and survival of colon cancer cells (Salari, Spulak et al., 2012). Homozygous loss of CDX2 led to inhibition of anchorage-independent growth which was confirmed in xenograft studies (Dang, Chen et al., 2006). Loss of CDX2 has been suggested to be an infrequent event during the development of CRC (Witek, Nielsen et al., 2005). However, there are also evidence in literature indicating that CDX2 can act as a tumor suppressor Several studies have been performed in different models that all concluded that the reduction or the loss of CDX2 in the gut epithelium facilitates tumour growth, either cell-autonomously and/or non-cell-autonomously (Aoki, Kakizaki et al., 2011, Aoki, Tamai et al., 2003, Balbinot et al., 2018, Bonhomme, Duluc et al., 2003, Hryniuk, Grainger et al., 2014, Sakamoto, Feng et al., 2017). In colon cancer patients CDX2 expression is reported to be reduced (Bakaris, Cetinkaya et al., 2008, Kaimaktchiev, Terracciano et al., 2004), and loss of CDX2 has been suggested to be a poor prognostic marker of the disease (Aasebo, Dragomir et al., 2020). While the inventors cannot explain such divergent results, they believe that the lack of CDX2 mutations in cancer patients and absence of any deficiency in its gene expression levels (Wicking, Simms et al., 1998, Woodford-Richens, Halford et al., 2001) makes it likely that CDX2 functions as a oncogene or tumor suppressor in a context dependent manner.

In an effort to understand how DDSMs carry out their biological functions in the cells—it was essential to identify and validate their common targets. Present inventors intentionally narrowed down the search for targets with known functions in DNA damage recognition, signaling and repair. While four targets (BRCA1, ATM, Chk1 and RNF8) were initially validated, further characterization was done with BRCA1 to show modulation of BRCA1 levels have opposite effects on the levels of DNA repair, DNA damage and invasion using cell-based assays (FIG. 5, S5). It is interesting to note that BRCA1 and ATM are targeted by multiple miRs. For example, BRCA1 is downregulated by miR-182 (which is also one of the DDSMs) (Moskwa, Buffa et al., 2011), miR-1255b, miR-148b*, miR-193b* (Choi, Pan et al., 2014), miR-146a, miR-146b-5p (Garcia, Buisson et al., 2011) and miR-498 (Matamala, Vargas et al., 2016). Similarly ATM is targeted by miR-421 (Hu, Du et al., 2010), miR-101 (Yan, Ng et al., 2010) and miR-203 (Zhou, Wan et al., 2014). The question which maybe asked is why the DDSMs are different than the published literature with respect to ATM and BRCA1. The inventors believe that this divergence is because of a few unique reasons—the miRs had to be upregulated by specific types of DNA damage, had to have CDX2 binding site(s) in the promoter and be upregulated in colon cancer cells. Finally, the inventors believe that multiple miRs constitute the DDSMs because the biological system recognizes the importance of regulating an important process like DDR. The DDSMs bind to neighboring yet discrete sites on targets (like BRCA1, Figure S5O) and possibly carry out cooperative repression in vivo (Broderick, Salomon et al., 2011, Grimson, Farh et al., 2007, Saetrom, Heale et al., 2007). This also might be the reason why many of the DDSMs have neighboring non-canonical binding sites in the 3′UTR of the target genes like BRCA1.

As regards how DDSMs remain inactive and are thereby not expressed in the normal cells. The inventors provide evidence that these miRs are not transcribed as their common upstream effector, CDX2, is repressed due to the BLM-dependent recruitment of HDAC1/2 containing Sin3b and NuRD repressor complexes onto the promoter (FIG. 6, S7). In unsynchronized cycling cells BLM is known to be present throughout the nucleoplasm and in PML bodies (Sanz, Proytcheva et al., 2000) while after exposure to DNA damage (like HU or IR), BLM relocates to the sites of damage (Sengupta et al., 2004, Tripathi et al., 2018) and is thought to regulate both NHEJ and HR in a cell cycle specific manner (Tripathi et al., 2018). It has been proposed that BLM functions as a tumor suppressor only after exposure to exogenous stress. The inventors propose that even in unstressed cells the nucleoplasmic BLM has tumor suppressive function by taking up the role of an adaptor protein. In this process BLM brings the HDAC1 and HDAC2 containing repressor complexes (Sin3b and NuRD) in contact with the transcription factors (like SMAD3) bound to the CDX2 promoter and thereby represses its transcription. Interestingly the adaptor function of BLM has been reported earlier whereby it helps in the interaction of c-Jun and c-Myc oncoproteins to their common E3 ligase, Fbw7α, and thereby facilitating the degradation of the substrates (Chandra, Priyadarshini et al., 2013, Priyadarshini, Hussain et al., 2018). Interestingly, after DNA damage, when BLM is no longer nucleoplasmic and instead gets redistributed to the site of damage, the repression of CDX2 promoter is reversed. Most probably BLM acts in conjunction or in coordination with other factors have also been reported to recruit HDAC1 and HDAC2 containing NuRD complex to the CDX2 promoter (Graule, Uth et al., 2018, Shakya, Kang et al., 2011, Yuri, Fujimura et al., 2009). The binding of BLM to the CDX2 promoter in the normal tissues, thereby keeping it inactive (FIG. 6P), subsequently results in the low levels of miRs (FIG. 7A-7D) and consequently high level of BRCA1 in both tissues and blood samples of the patients (Figure S8). These results provide evidence that the DDSMs are biologically active moieties present in the colon cancer patient tissue samples. These results also add to the mechanistic reasons for the decrease in BRCA1 and ATM levels seen in colon cancer tissues samples which correlated with their reduced overall survival (Bai, Tong et al., 2004, Grabsch, Dattani et al., 2006, Wang, Zhao et al., 2018).

If DDSMs respond to the extent of DNA damage and have an oncogenic role, it maybe possible to revert the process of neoplastic transformation by inhibiting the miRs. Indeed, using two different experimental strategies inhibition of the DDSMs led to the regression of tumors (FIG. 2F, 2I). Similar approaches to target tumors using either RNA aptamers or chemical ligands have also been reported (Shu, Pi et al., 2014). Other complementary approaches, like the usage of miRNA sponges, miRNA masking, antisense oligonucleotides or small molecule inhibitors (Hernandez, Sanchez-Jimenez et al., 2018), would perhaps give an even better effect. The low levels of BRCA1 and ATM in cells having overexpressed DDSMs indicates defective DNA damage response and homologous recombination pathways. The inventors suggest that colon cancer patients with increased DDSMs in tissues and/or blood should be treated with PARP inhibitors like olaparib and veliparib, as already being proposed in certain studies (Clark et al., 2012, Davidson et al., 2013, Wang et al., 2017).

In conclusion and as discussed above, the identification of DDSMs has implication as it is perhaps for the first time an entire workflow has been obtained for the functioning of a cancer specific miR signature—whereby the identity of the upstream regulator (CDX2) and the downstream effectors (DDR proteins like BRCA1, ATM, Chk1 and RNF8) have been simultaneously elucidated and validated in both mice models and patient samples. Further, this invention opens up the possibility of the usage of the DDSMs as a potential cancer prognostic biomarker, as a target for miR inhibition and usage of synthetic lethality as a treatment procedure—all attractive avenues of future research.

In this regard, the present invention is providing a microRNA (miR) signature also called DNA damage sensitive miRs (DDSMs) which responds to the levels of DNA damage. The present invention provides method for identification of DDSMs which are upregulated by a common transcription factor (CDX2). Further, the present invention also provides miR inhibitors along nanoparticle delivery system for administering the same, nucleic acid constructs, expression cassettes, vectors and kits comprising the same. The microRNA (miR) signature of the present invention can be used as biological markers to detect earliest stages of cancer. The present invention also provides a method of treatment of other types of cancer and related diseases.

Further, the kit of the present invention is able to detect the levels of the DDSMs and BRCA1 in the tissues and blood of the colon cancer patients.

### EXAMPLES

The following examples serve to illustrate certain embodiments and aspects of the present disclosure and are not to be considered as limiting the scope thereof.

### Methods

### Antibodies, Plasmids, siRNAs

The antibodies used in the study have been listed in Table S3A below:

TABLE S3A
List of antibodies used in the study

| Name of antibody | Source | Identifier |
|---|---|---|
| Anti-Flag (used for | Sigma-Aldrich | Cat# F1804: RRID:AB_262044 |
| WB) | | |
| Anti-Flag M2 | Sigma-Aldrich | Cat# F2220 |
| affinity gel (used for | | |
| IP) | | |
| Anti-BLM | Bethyl Laboratories | Cat# A300-110A; RRID: |
| (used for WB, ChIP) | | AB_2064794 |
| Anti-CDX2 | Abcam | Cat# ab76541; RRID: AB_1523334 |
| (used for WB, IF, | | |
| EMSA, IHC) | | |
| Anti-CDX2 | Santa Cruz Biotechnology | Cat# sc-134468; RRID: AB_2260275 |
| (used for WB) | | |
| Anti-CDX2 | Santa Cruz Biotechnology | Cat# sc-166830; RRID: AB_2260278 |
| (used for WB) | | |
| Anti-BRCAI | Abcam | Cat# ab16780; RRID: AB_2259338 |
| (used for WB, IHC) | | |
| Anti-GFP | Santa Cruz Biotechnology | Cat# sc-9996; RRID:AB_627695 |
| (used for WB. IF) | | |
| Anti-hsp90 | Santa Cruz Biotechnology | Cat# sc-7947; RRID:AB_2121235 |
| (used for WB) | | |
| Anti-PCNA | Santa Cruz Biotechnology | Cat# sc-56; RRID:AB_628110 |
| (used for WB) | | |
| Anti-RAD51 | Santa Cruz Biotechnology | Cat# sc-8349; RRID: AB_2253533 |
| (used for WB) | | |
| Anti-H2AX | Abcam | Cat# ab81299: RRID:AB_1640564 |
| (used for WB. IF) | | |
| Anti-53BPI | BD Biosciences | Cat# 612523; RRID:AB_399824 |
| (used for IF) | | |
| Anti-CD31 | Abcam | Cat# ab28364; RRID:AB_726362 |
| (used for IHC) | | |
| Anti-ATM | Santa Cruz Biotechnology | Cat# sc-377293 |
| (used for WB) | | |
| Anti-Chk1 | Cell Signaling Technology | Cat# 2360; RRID:AB_2080320 |
| (used for WB) | | |
| Anti-RNFS | Santa Cruz Biotechnology | Cat# sc-271462; RRID:AB_10648902 |
| (used for WB) | | |
| Anti-HA probe | Santa Cruz Biotechnology | Cat# sc-7392; RRID:AB_627809 |
| (used for WB) | | |
| Anti-SMAD3 | Abcam | Cat# ab28379: RRID:AB_2192903 |
| (used for ChIP, WB) | | |
| Anti-Sin3b | Santa Cruz Biotechnology | Cat# sc-13145; RRID:AB_628254 |
| (used for ChIP, IP, | | |
| WB) | | |
| Anti-AP2B | Santa Cruz Biotechnology | Cat# sc-390119 |
| (used for ChIP) | | |
| Anti-HDAC1 | Abcam | Cat# ab7028; RRID:AB_305705 |
| (used for ChIP, WB) | | |
| Anti-HDAC2 | Abcam | Cat# ab7029: RRID:AB_305706 |
| (used for ChIP, WB) | | |
| Anti-CHD4 | Abcam | Cat# ab70469; RRID:AB_2229454 |
| (used for ChIP, WB) | | |

WB: Westem blotting
IP: Immunoprecipitation
IF: Inununofluorescence
IHC. Innnunohistochemistry
EMSA: Electrophoretic Mobility Shift Assay
ChIP: Chromatin Immunoprecipitation Assay

The recombinants listed in Table S3B:

TABLE S3B
List of recombinant DNAs used in the study

| Name of the recombinant | | |
|---|---|---|
| DNA | Source | Identifier |
| CMV-β gal | Present in the lab of | N/A |
| | corresponding author | |
| pcDNA3 Flag BLM (1-1417) | Present in the lab of | (Kharat, Tripathi et |
| | corresponding author | al., 2016) |
| pFlag CDX2 | Jean-Noel Freund (Universite de | (Balbinot, Vanier et |
| | Strasbourg, France) | al., 2017) |
| pFlag CDX2 mini | Jean-Noel Freund (Universite de | (Balbinot et al., |
| | Strasbourg, France) | 2017) |
| pGEX4T-1 CDX2 | This study | N/A |
| CMV24 3XFlag CDX2 WT | This study | N/A |
| CMV24 3XFlag CDX2 R190A | This study | N/A |
| CMV24 3XFlag CDX2 R238A | This study | N/A |
| CMV24 3XFlag CDX2 R240A | This study | N/A |
| pGL3-miR-96/182/183 WT | This study | N/A |
| promoter luc | | |
| pGL3-miR-96/182/183 MT | This study | N/A |
| promoter luc | | |
| pGL3-miR-378a-3p WT | This study | N/A |
| promoter luc | | |
| pGL3-miR-561-5p WT | This study | N/A |
| promoter luc | | |
| pGL3-miR-584-5p WT | This study | N/A |
| promoter luc | | |
| pGL3-miR-584-5p MT | This study | N/A |
| promoter luc | | |
| pGL3-BRCA1 3'UTR miR- | This study | N/A |
| 183 WT | | |
| pGL3-BRCA1 3'UTR miR | This study | N/A |
| 378a-3p WT | | |
| pGL3-BRCA1 3'UTR miR | This study | N/A |
| 584-5p WT | | |
| pGL3-BRCA1 3'UTR miR- | This study | N/A |
| 183 MT | | |
| pGL3-BRCA1 3'UTR miR | This study | N/A |
| 378a-3p MT | | |
| pGL3-BRCA1 3'UTR miR | This study | N/A |
| 584-5p MT | | |
| pcDNA3 HA-BRCAI | Dipanjan Chowdhury (Dana- | N/A |
| | Farber Cancer Institute. USA) | |
| EGFP-C1-BLM | Nathan Ellis (University of | (Hu, Beresten et al., |
| | Arizona, USA) | 2001) |
| pcDNA3 Sin3b Flag | Gregory David (New York | (Bainor, Saini et al., |
| | University School of Medicine, | 2018) |
| | USA) | |
| pTRIPZ shSin3b | Gregory David (New York | (Bainor et al., 2018) |
| | University School of Medicine, | |
| | USA) | |
| pTRIPZ sh Scrambled | Gregory David (New York | (Bainor et al., 2018) |
| | University School of Medicine, | |
| | USA) | |
| Flag-HDAC1 | Present in the lab of | N/A |
| | corresponding author | |
| GST-HDAC1 | Gordon Hager (National Cancer | (Qiu, Stavreva et al., |
| | Institute, National Institutes of | 2011) |
| | Health. USA) | |
| pCMVSB-Flag-SMAD3 | Jeff Wrana (University of | (Labbe, Silvestri et |
| | Toronto, Canada) via Addgene | al, 1998) |
| | (Cat# 11742) | |
| pGEX6-SMAD3 WT | Joan Massague (Slaon Kettering | (Gao, Alarcon et al., |
| | Institute, USA) via Addgene | 2009) |
| | (Cat# 27010) | |
| pLenti-III-mir-Off Control | Abm Inc. | Cat# m007 |
| Vector | | |

And the reagents used are in Table S3C:

TABLE S3C
List of reagents used in the study

| | | |
|---|---|---|
| Name | Source | Identifier |

Chemicals

| | | |
|---|---|---|
| IPTG | Sigma-Aldrich | Cat# 16758; CAS Number |
| | | 367-93-1 |
| PMSF | Sigma-Aldrich | Cat# P7626: CAS Number |
| | | 329-98-6 |
| DTT | Sigma-Aldrich | Cat# D0632; CAS Number |
| | | 3483-12-3 |
| Triton-X-100 | Sigma-Aldrich | Cat# T9284; CAS Number |
| | | 9002-93-1 |
| Hydroxyurea | Sigma-Aldrich | Cat# H8627: CAS Number |
| | | 127-07-1 |
| Puromycin | Sigma-Aldrich | Cat# P8833; CAS Number |
| | | 58-58-2 |
| G418 Sulfate | Sigma-Aldrich | Cat# A1720; CAS Number |
| | | 108321-42-2 |
| Hygromycin | Sigma-Aldrich | Cat# H3274; CAS Number |
| | | 31282-04-9 |
| Giemsa stain | Sigma-Aldrich | Cat# G5637; CAS Number |
| | | 51811-82-6 |
| Toluidine Blue | Sigma-Aldrich | Cat# 89640; CAS Number |
| | | 6586-04-5 |
| Crystal Violet | Sigma-Aldrich | Cat# C0775; CAS Number |
| | | 948-62-9 |
| Zeocin | Thermo Fisher Scientific | Cat# R25005; CAS Number |
| | | 11031-11-1 |
| Blasticidine S | Sigma-Aldrich | Cat# 15205; CAS Number |
| hydrochloride | | 3513-03-9 |
| Luciferin | Sigma-Aldrich | Cat# L9504; CAS Number |
| | | 2591-17-5 |
| ONPG | Sigma-Aldrich | Cat# N1127; CAS Number |
| | | 369-07-3 |

Recombinant proteins

| | | |
|---|---|---|
| GST HDACI (WT) | This study | N/A |
| GST SMAD3 (WT) | This study | N/A |
| GST CDX2 (WT) | This study | N/A |

Cell Lines

| | | |
|---|---|---|
| HEK293T | ATCC | Cat# CRL-3216 |
| Lenti-X 293T | Clontech | Cat# 632180 |
| GM03509 GFP-BLM | Present in the lab of | (Kharat et al., 2016) |
| Clone 4.3.4 | corresponding author | |
| GM03509 GFP Clone | Present in the lab of | (Kharat et al, 2016) |
| 100 | corresponding author | |
| GM08505 GFP-BLM | Nathan Ellis (The University of | (Hu et al., 2001) |
| | Arizona Cancer Center, USA) | |
| GM08505 GFP | Nathan Ellis (The University of | (Hu et al., 2001) |
| | Arizona Cancer Center, USA) | |
| SW480 | National Cell Repository. | N/A |
| | NCCS, Pune | |
| HeLa S3 Flag AGO2 | Annick Harel-Bellan | (Noune, Ameyar-Zazoua et |
| | (Institut de Hautes Etudes | al., 2010) |
| | Scientifiques, University of | |
| | Paris-Saclay, France) | |
| HeLa S3 pREV | Annick Harel-Bellan | (Nonne et al., 2010) |
| | (Institut de Hautes Etudes | |
| | Scientifiques, University of | |
| | Paris-Saclay, France) | |
| HCT116 WT | Bert Vogelstein (Johns | (Traverso, Bettegowda et |
| | Hopkins Medicine, USA) | al., 2003) |
| HCTI16 BLM -/- | Bert Vogelstein (Johns | (Traverso et al., 2003) |
| | Hopkins Medicine, USA) | |
| HCT116 BLM -/- Inhi | This study | N/A |
| Control | | |
| HCT116 BLM -/- Inhi | This study | N/A |
| 96-5p | | |
| HCT116 BLM -/- Inhi | This study | N/A |
| 29a-5p | | |
| HT29 Dox inducible | Isabelle Gross (Universite de | (Gross, Duluc et al., 2008) |
| CDX2/GFP (TW6) | Strasbourg, France) | |
| HT29 Dox inducible | Isabelle Gross (Universite de | (Gross et al., 2008) |
| GFP (TG8) | Strasbourg, France) | |
| HCT116 CDX2 (HW2) | Isabelle Gross (Universite de | N/A |
| | Strasbourg, France) | |
| HCT116 Vector (HC1) | Isabelle Gross (Universite de | N/A |
| | Strasbourg, France) | |

Oligonucleotides

| | | |
|---|---|---|
| siRNA sequences for | Dharmacon | (Bai, Ye et al., 2013) |
| CDX2 | | |
| siRNA sequences for | Dharmacon | (Tikoo. Madhavan et al .. |
| BLM | | 2013) |
| siRNA sequences for | Dharmacon | (Coene, Hollinshead et al., |
| BRCAI | | 2005) |
| siRNA sequences for | Dharmacon | (Nio, Yamashita et al., |
| CHD4 | | 2015) |
| siRNA sequences for | Dharmacon | (Palmisano, Della Chiara et |
| HDACI | | al., 2012) |
| siRNA sequences for | Dharmacon | (Palmisano et al., 2012) |
| HDAC2 | | |
| ON-TARGETplus Non- | Dharmacon | Cat# D-001810-01-05 |
| targeting siRNA #1 | | |
| miR-mimic control | Sigma-Aldrich | Cat# HMC0002 |
| miR-mimic 29a-5p | Sigma-Aldrich | Cat# HMMI0433 |
| miR-mimic 29b-3p | Sigma-Aldrich | Cat# HMI0438 |
| miR-mimic 96-5p | Sigma-Aldrich | Cat# HMI0980 |
| miR-mimic 139-5p | Sigma-Aldrich | Cat# HMI0212 |
| miR-mimic 182-Sp | Sigma-Aldrich | Cat# HMI0275 |
| miR-mimic 183-5p | Sigma-Aldrich | Cat# HMI0280 |
| miR-mimic 335-3p | Sigma-Aldrich | Cat# HMI0491 |
| miR-mimic 378a-3p | Sigma-Aldrich | Cat# HMI0546 |
| miR-mimic 486-5p | Sigma-Aldrich | Cat# HMI0596 |
| miR-mimic 561-5p | Sigma-Aldrich | Cat# HMI2158 |
| miR-mimic 584-5p | Sigma-Aldrich | Cat# HMI0807 |
| miR-inhibitor control | Sigma-Aldrich | Cat# NCSTUD001 |
| miR-inhibitor-29a-5p | Sigma-Aldrich | Cat# HSTUDO433 |
| miR-inhibitor-29b-3p | Sigma-Aldrich | Cat# HSTUDO436 |
| miR-inhibitor-96-5p | Sigma-Aldrich | Cat# HSTUD0980 |
| miR-inhibitor-139-5p | Sigma-Aldrich | Cat# HSTUDO212 |
| miR-inhibitor-182-5p | Sigma-Aldrich | Cat# HSTUDO275 |
| miR-inhibitor-183-5p | Sigma-Aldrich | Cat# HSTUDO280 |
| miR-inhibitor-335-3p | Sigma-Aldrich | Cat# HSTUDO491 |
| miR-inhibitor-378a-3p | Sigma-Aldrich | Cat# HSTUDO546 |
| miR-inhibitor-486-5p | Sigma-Aldrich | Cat# HSTUDO596 |
| miR-inhibitor-561-5p | Sigma-Aldrich | Cat# HSTUD2095 |
| miR-inhibitor-584-5p | Sigma-Aldrich | Cat# HSTUD2232 |
| RT-qPCR primers | Table S3D | N/A |
| ChIP primers for CDX2 | Table S3D | N/A |
| promoter | | |
| ChIP primers for | Table S3D | N/A |
| GAPDH promoter | | |
| Primers for EMSA | Table S3D | N/A |
| Primers for measuring | Table S3D | N/A |
| levels of mature miRs | | |
| Primers for measuring | Table S3D | N/A |
| levels of precursor miRs | | |
| Primers for measuring | Table S3D | N/A |
| levels of primary miRs | | |
| Primers for measuring | Table S3D | N/A |
| levels of control RNA | | |

Other

| | | |
|---|---|---|
| Fetal bovine Serum | Thermo Fisher Scientific | Cat# 10082147 |
| Advanced DMEM | Thermo Fisher Scientific | Cat# 12491-023 |
| QuikChange II XL Site- | Agilent | Cat# 200522 |
| Directed | | |
| Mutagenesis Kit | | |
| T7 Quick coupled | Promega | Cat# L2080 |
| Transcription | | |
| Translation system | | |
| [S] Methionine | Perkin Elmer | Cat# NEG009T |
| [x 2P] ATP | Perkin Elmer | Cat# BLU002Z250UC |
| Trizol reagent | Thenno Fisher Scientific | Cat# 15596026 |
| Trizol LS reagent | Thermo Fisher Scientific | Cat# 10296010 |
| Reverse Transcriptase | Eurogentec | Cat# RT-RICK-05 |
| Core Kit | | |
| Qubit dsDNA HS assay | Thermo Fisher Scientific | Cat# Q32851 |
| kit | | |
| Lipofectamine 2000 | Thermo Fisher Scientific | Cat# 11668019 |
| Complete Protease | Roche | Cat# 11697498001 |
| Cocktail inhibitor | | |
| BL21-CodonPlus-RP | Agilent | Cat# 230250 |
| Poly-Prep | Biorad | Cat# 73101550 |
| Chromatography column | | |
| Flag peptide | Sigma-Aldrich | Cat# 3290 |
| Long Amp Taq DNA | New Englands Biolab | Cat# M0323L |
| polymerase | | |
| QIAamp DNA Mini Kit | Qiagen | Cat# 51304 |
| DyNAmo ColorFlash | Thermo Fisher Scientific | Cat# F416L |
| SYBR Green qPCR Kit | | |
| LentimiRa-Off-hsa-miR- | Abm Inc. | Cat# mh36217 |
| 96-5p Virus | | |
| LentimiRa-Off-hsa-miR- | Abm Inc. | Cat# mh35398 |
| 29a-5p Virus | | |
| Lenti-X HTX Packaging | Takara | Cat# 631249 |
| System | | |
| LNA TM Universal RT | Exiqon | Cat# E23301 |
| miRNA PCR kit. 8-64 | | |
| rxnS | | |
| mIRCURY LNA RT Kit | Qiagen | Cat# 339340 |
| Superscript II Reverse | Thermo Fisher Scientific | Cat# 18064022 |
| Transcriptase | | |
| 6% Novex TBE PAGE | Thermo Fisher Scientific | Cat# EC6265BOX |
| Gel | | |

Animals

| | | |
|---|---|---|
| NOD SCID mice | The Jackson Laboratory | Stock# 001303 |

pGEX4T-1 CDX2 was obtained by cloning full-length CDX2 into BamH1 and EcoR1 sites of pGEX4T-1, CMV24 3× Flag CDX2 WT was generated by cloning the corresponding insert into the HindIII and KpnI sites of CMV24. For cloning the CDX2 binding site(s) containing miR promoters into pGL3 vector the following cloning sites were used: pGL3-miR-96/182/183 WT promoter luc (KpnI and HindIII), pGL3-miR-378a-3p WT promoter luc (KpnI and HindIII), pGL3-miR-561-5p WT promoter luc (NheI and HindIII), pGL3-miR-584-5p WT promoter luc (KpnI and HindIII). The BRCA1 3′ UTR sequences containing the miR binding sequences were inserted into the KpnI and HindIII sites to generate pGL3-BRCA1 3′UTR miR-183 WT, pGL3-BRCA1 3′UTR miR 378a-3p WT, pGL3-BRCA1 3′UTR miR 584-5p WT. All site-directed mutagenesis was carried out using QuikChange II XL Site-Directed Mutagenesis Kit. The siRNA and shRNA sequences used were: CDX2 (AAC CAG GAC GAA AGA CAA AUA), BLM (AGC AGC GAU GUG AUU UGC A), BRCA1 (UCA CAG UGU CCU UUA UGU A), CHD4 (CCC AGA AGA GGA UUU GUC A), HDAC1 (GGC UCC UAA AGU AAC AUC AUU), HDAC2 (CCA CCA UGC UUU AUG UGA UUU) and Sin3b (AGG CUG UAG ACA UCG UCC A).

### Cells

All pre-existing cell lines (Table S3C) were maintained as described in the original publications or as recommended by the suppliers. HC1, HW7 cells were generated by stably transfecting HCT116 cells pCB6 (for HC1) or pCB6-Flag₂-mCDX2 WT (for HW7) and selecting with G418. The cells were grown in DMEM+10% FBS+G418 (1 mg/ml)+antibiotics. To generate the HCT116 BLM−/− Inhi Control cells, pLenti-III-mir-Off Control Vector (Abm Inc.) was used. The lentivirus was generated by using Lenti-X HT packaging mix in Lenti-X 293T. To obtain HCT116 BLM−/− Inhi-29a-5p and HCT116 BLM−/− Inhi-96-5p lines, commercial lentivirus particles were used (Abm Inc.). BLM−/− cells were plated in six well cluster and transduced with the three different lentiviral particles. Transduction was carried out with 2 μg/ml polybrene. Medium was changed 24 hours post-transduction. For selection, 1 μg/ml puromycin was added to the cells. The transduced cells were grown in presence of 1 μg/ml puromycin for stable line generation for 7 days after which the clones were analysed for the expression of the two miRs.

### Example 1: Identification of a DNA Damage Dependent miR Signature

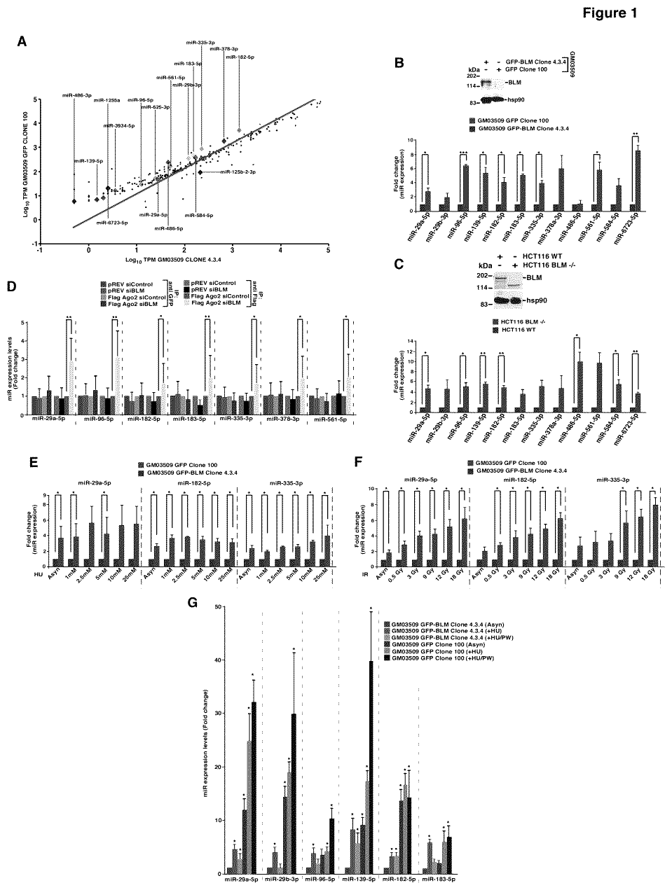

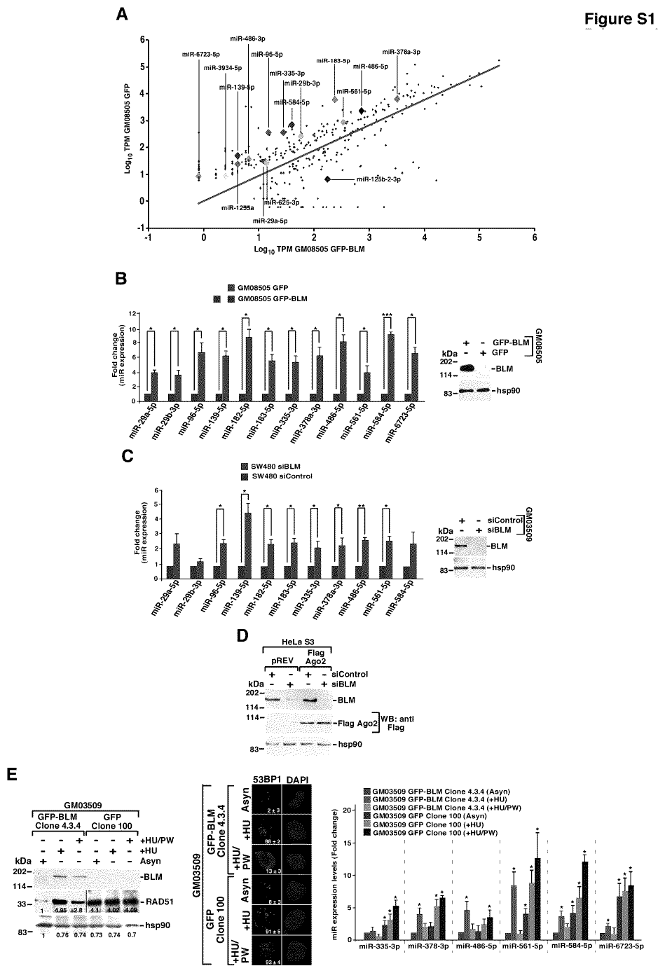

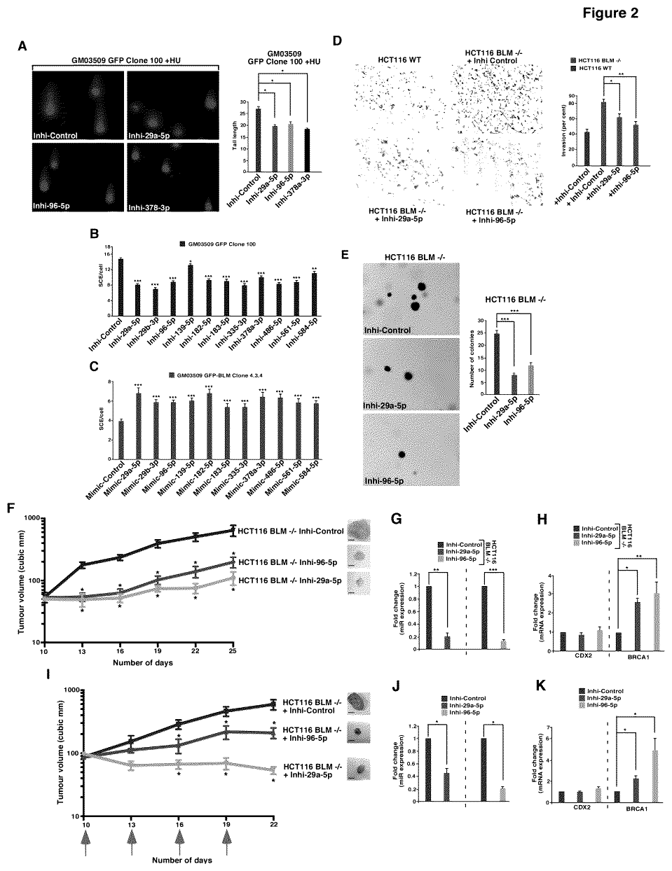

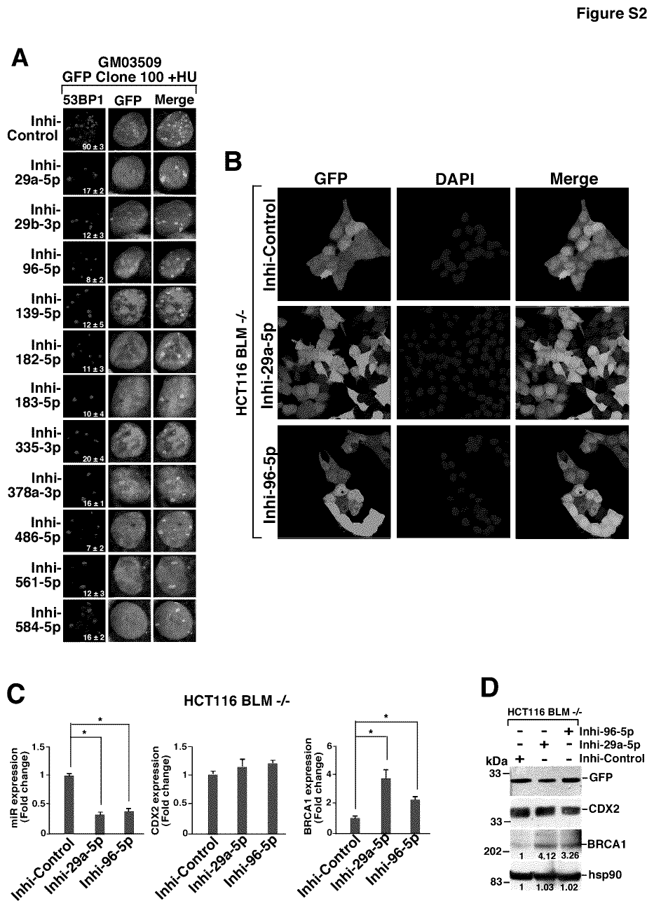

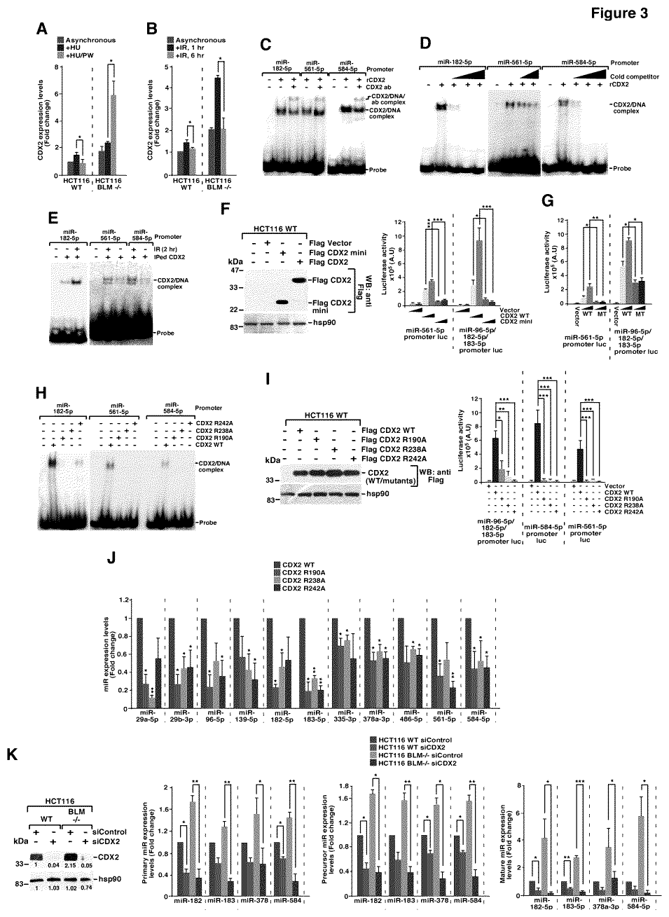

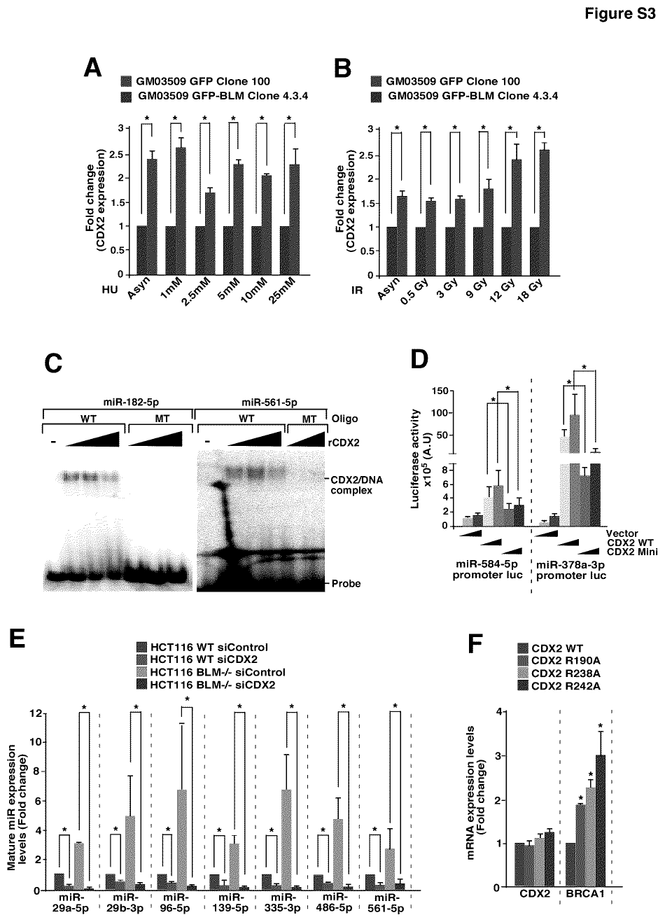

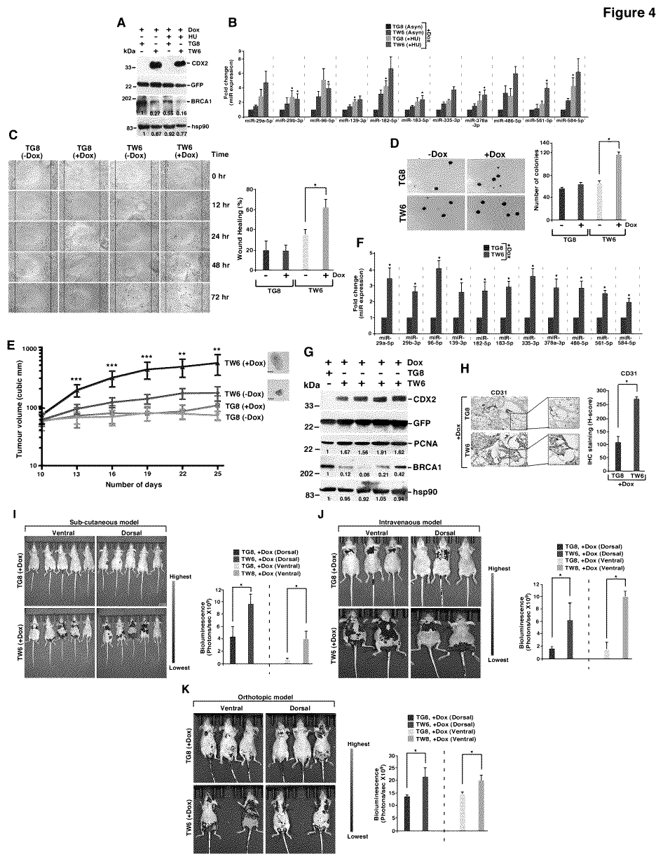

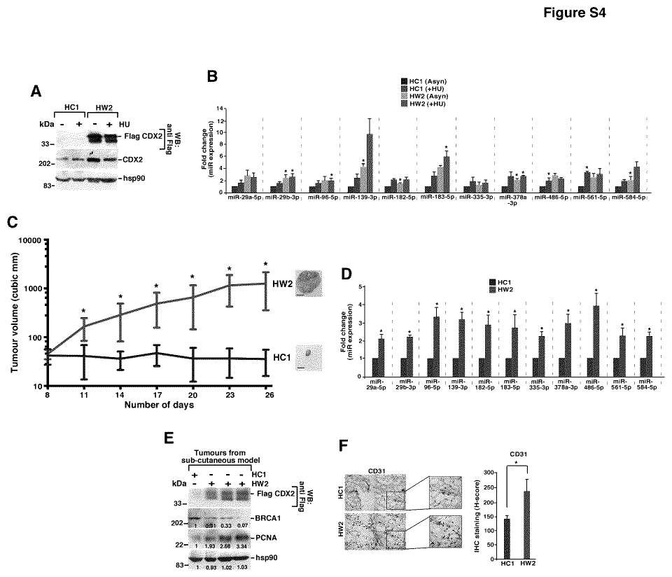

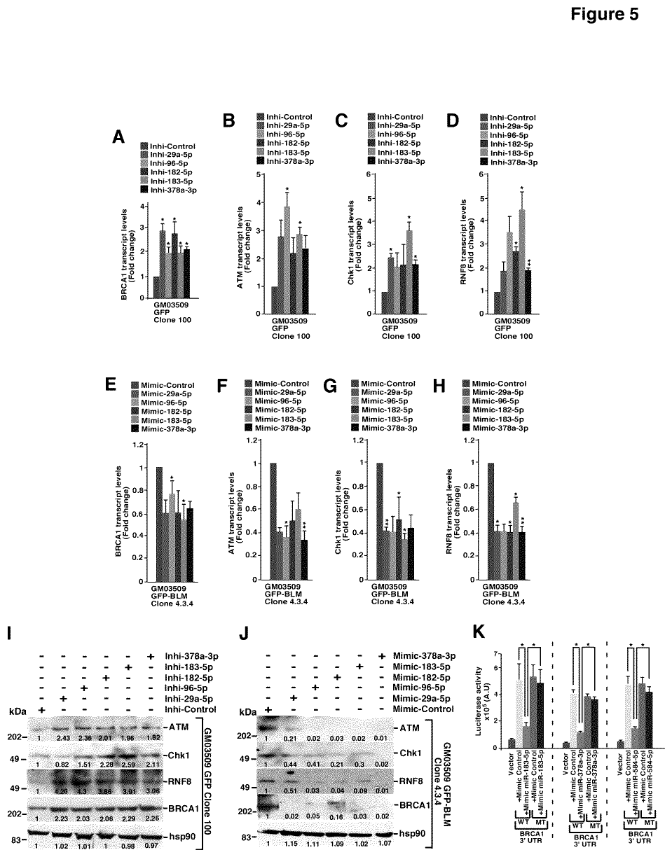

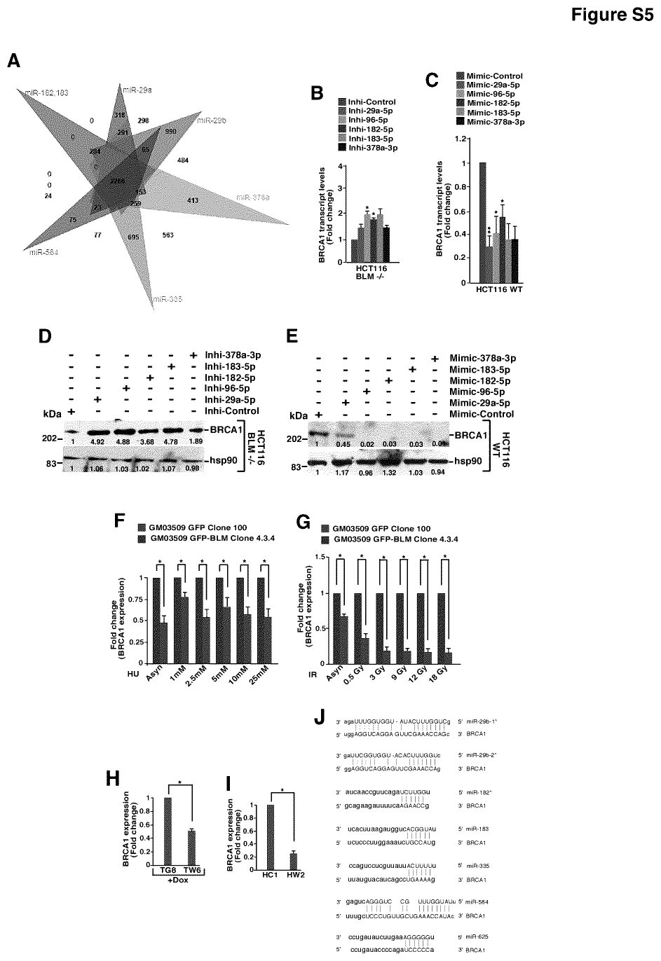

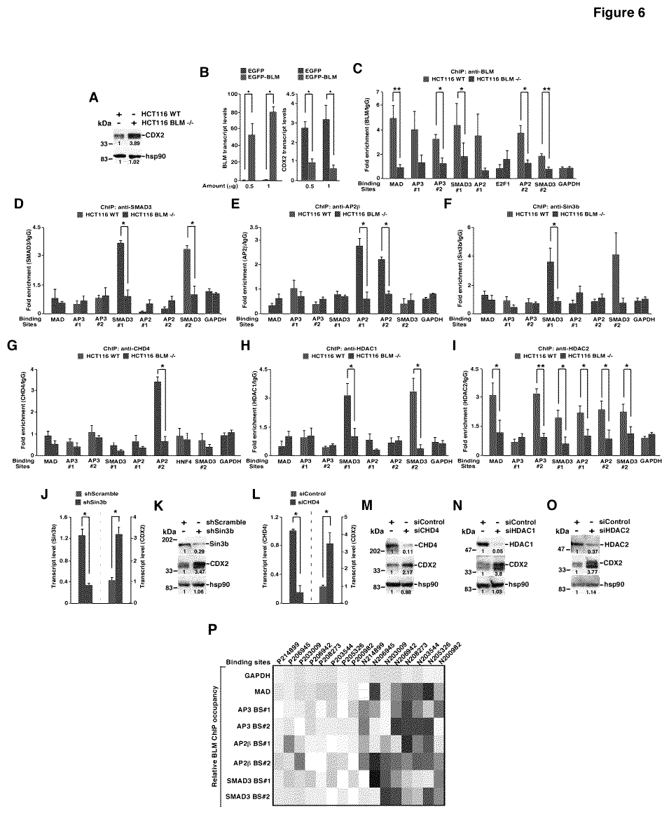

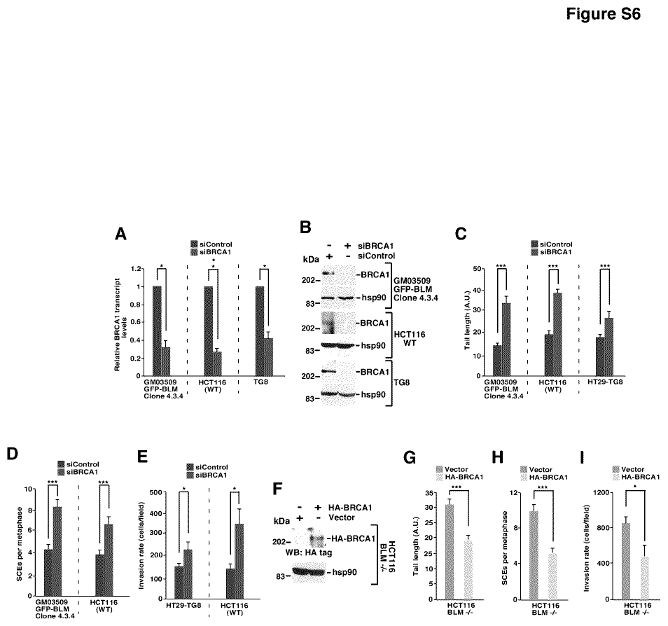

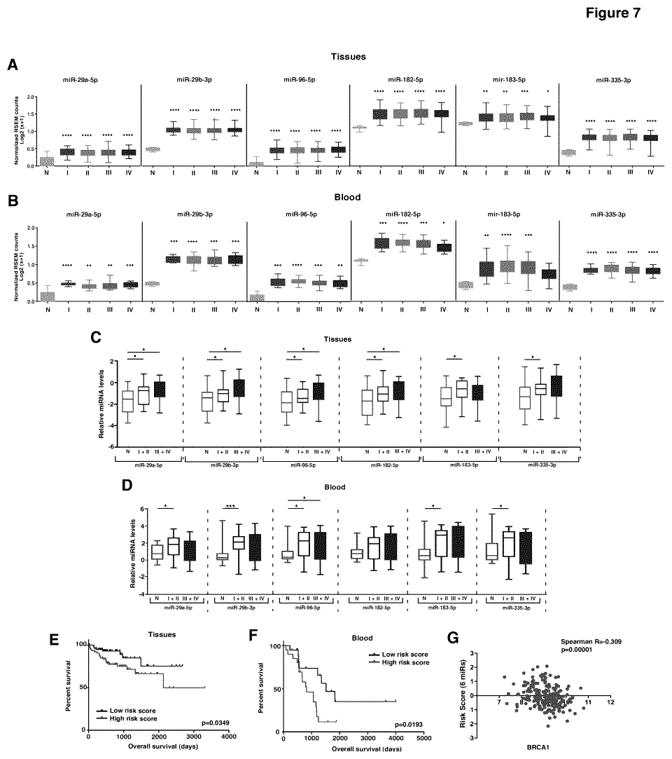

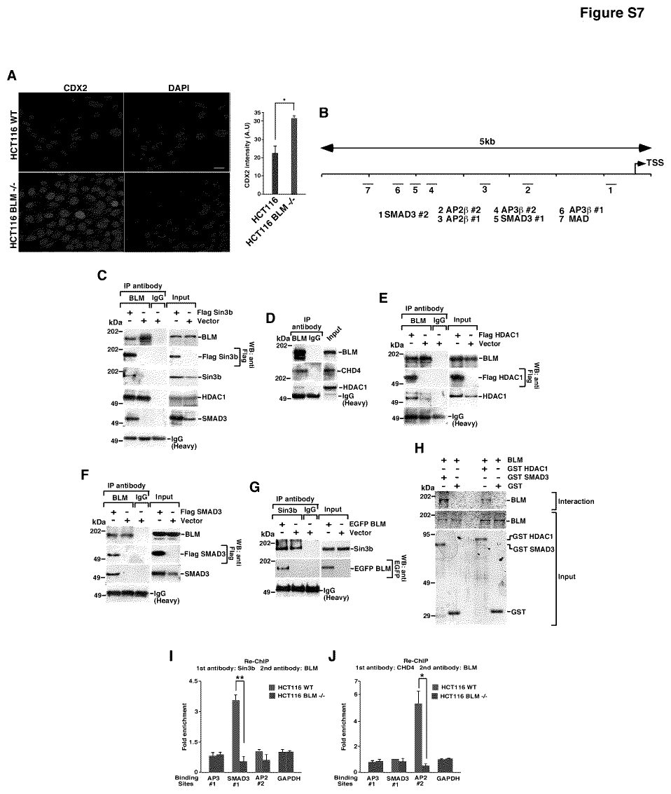

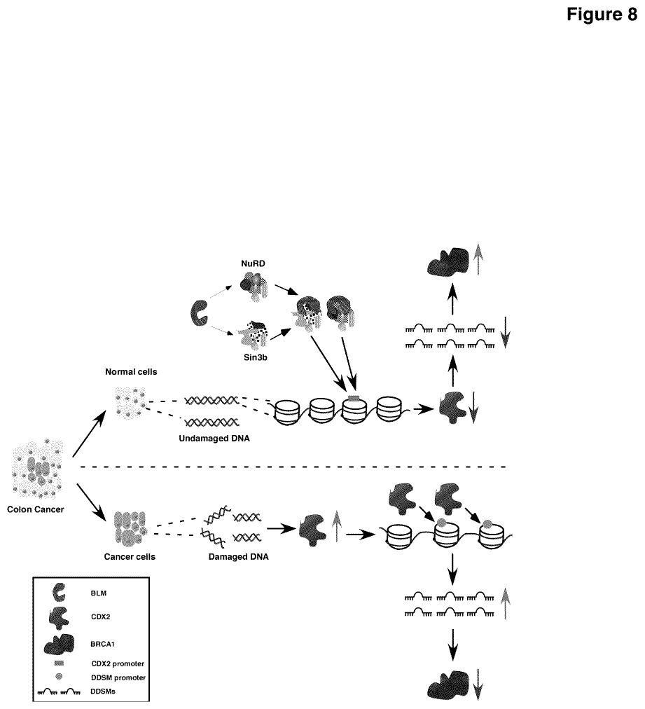

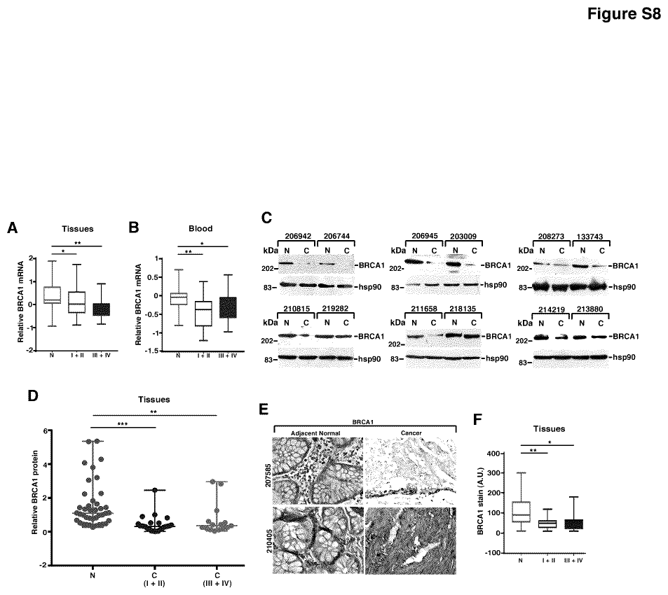

In trying to understand how DNA damage modulates miR expression, the inventor identified the miRs whose expression levels were increased in presence of high levels of endogenous damage. For that purpose, the inventor carried out small RNA sequencing with RNA isolated from two pairs of isogenic cell lines created from two BS patients—immortalized cells from GM03509 complemented with either GFP-BLM (Clone 4.3.4) or GFP (Clone 100) (FIG. 1A) or immortalized cells from another BS patient GM08505 complemented with either GFP-BLM or GFP (Figure S1A). Sixteen miRNAs were significantly up-regulated in absence of BLM in both the above isogenic pairs while one miR was down-regulated in absence of BLM expression. The inventors focused on the miRs which were upregulated in the absence of BLM in both the pairs of the cell lines. Using RT-qPCR, the inventors validated the relative expression of these miRs in the same isogenic pairs of cells (FIG. 1B, SIB). Additionally, the inventors validated the increased expression of these miRs in two more isogenic lines of colon cancer origin—one of the most common cancers in BS patients. The inventors found that lack of BLM in both SW480 and HCT116 cells led to increased expression of these miRs (FIG. 1C, SIC). The inventors named these sixteen miRs (SEQ ID. NO. 1-miR-29a-5p, SEQ ID. NO. 2-miR-29b-3p, SEQ ID. NO. 3-miR-96-5p, SEQ ID. NO. 4-miR-139-5p, SEQ ID. NO. 5-miR-182-5p, SEQ ID. NO. 6-miR-183-5p, SEQ ID. NO.7-miR-335-3p, SEQ ID. NO. 8-miR-378a-3p, SEQ ID. NO. 9-miR-486-3p, SEQ ID. NO. 10-miR-486-5p, SEQ ID. NO. 11-miR-561-5p, SEQ ID. NO. 12-miR-584-5p, SEQ ID. NO. 13-miR-625-3p, SEQ ID. NO. 14-miR-1255a, SEQ ID. NO. 15-miR-3934-5p, SEQ ID. NO. 16-miR-6723-5p) as DNA damage sensitive miRs (DDSMs).

Next, the inventors wanted to determine if the matured DDSMs were also enriched in the RISC under the same conditions. Hela S3 cells stably integrated with either vector pREV or Flag Ago2 were used for the experiment. BLM was shut down in both the cell lines using BLM siRNA. In either absence or presence of BLM, immunoprecipitations were carried out with either anti-GFP (which acted as an antibody control) or with anti-Flag antibody. RNA bound to the immunoprecipitated Ago2 complex was isolated followed by RT-qPCR which showed that all the seven tested DDSMs were associated with Ago2 protein (i.e. the RISC complex) (FIG. 1D).

Absence of BLM leads to persistence of damaged DNA, which culminates in hyper-recombination (Croteau, Popuri et al., 2014, Tikoo & Sengupta, 2010). Hence, the inventors hypothesized that these miRs which were upregulated in absence of BLM may actually be responding to the increased levels of cellular DNA damage. To test this hypothesis, cell which express or lack BLM were exposed to a range of HU (leading to stalled replication forks) or IR (leading to DSB generation). The inventors found that the levels of the DDSMs increased over a range of HU concentrations (FIG. 1E) or IR dosages (FIG. 1F). Moreover, it was observed that in BLM+cells (GM03509 GFP-BLM Clone 4.3.4), the enhanced levels of DDSMs came back to near baseline levels when HU was washed off (as revealed by 53BP1 foci and RAD51 levels, Figure S1E, left and middle panels). In case of GM03509 GFP Clone 100 cells the oscillatory effect of the DDSMs was much less pronounced as substantial amount of DNA damage remained unrepaired as evident from the elevated protein levels of RAD51 and 53BP1 foci (FIG. 1G, Figure S1E).

### Example 2: Overexpression of DDSMs Cause Neoplastic Transformation

The levels of DDSMs depend on the extent of DNA damage and the persistence of intrinsic DNA damage leads to tumorigenesis (Bartek, Bartkova et al., 2007). Hence, the inventors next wanted to determine whether these miRs can induce neoplastic transformation in both in vitro and in vivo models. A direct correlation is known to exist between the extent of DNA damage, the lack of optimal levels of homologous recombination and the propensity of cells to undergo neoplastic transformation (Krajewska, Fehrmann et al., 2015, Li & Heyer, 2008). Compared to cells expressing BLM, cells which do not express BLM have high levels of intrinsic DNA damage which is also reflected in high rates of sister chromatid exchanges (SCEs) (Wilson & Thompson, 2007). The inventors found that the ablation of DDSMs in GM03509 GFP100 cells caused a decrease in the extent of residual DNA damage as determined by Comet assays (FIG. 2A), 53BP1 foci levels (Figure S2A), the spontaneous level of SCEs (FIG. 2B) while over-expression of the same DDSMs in GM03509 GFP-BLM Clone 4.3.4 cells led to an enhancement in SCEs (FIG. 2C). Subsequently, inhibition of the three DDSMs in HCT116 BLM KO cells culminated in decrease in the invasive potential of these cells, as seen using both matrigel (FIG. 2D) and soft agar (FIG. 2E) assays.

To determine whether modulation of the levels of DDSMs affect the ability to initiate and propagate tumors, the inventors generated three stable lines in HCT116 BLM−/− cells which expresses either a control inhibitor or specific inhibitors of miR-29a-5p or miR-96-

5p. Each of the stable lines, which also constitutively expressed EGFP (Figure S2B), were validated for the lowering of the levels of miR-29a-5p or miR-96-5p (Figure S2C, left). Xenograft assays were carried out in NOD SCID mice by subcutaneously injecting these cells in animals DDSMs and monitored for tumor development. Results indicated that inhibition of the two DDSMs decreased the rate of tumor growth (FIG. 2F). As a second in vivo assay, 100 cubic mm tumors were subcutaneously generated using HCT116 BLM−/− cells. A nanoparticle mediated delivery system was used to deliver miR inhibitor Control, miR inhibitor 29a-5p or miR inhibitor 96-5p directly to the base of the tumors every third day for 4 days. Tumor formation was decreased when miR inhibitor 29a-5p or miR inhibitor 96-5p were injected (FIG. 2I). Analysis of the levels of miR-29a-5p and miR-96-5p in these tumors (excised at the end point in both the experiments) confirmed the decrease in the levels of the two miRs (FIG. 2G, 2J). Thus, the DDSMs are oncogenic miRs which promote neoplastic transformation.

### Example 3: CDX2 Regulates DNA Damage Dependent miRs

Next, the inventors wanted to determine how the DDSMs were regulated in the cellular milieu. Using an in silico approach the inventors first analyzed the upstream 5 kb promoters of each of these miRs. The inventors found that only one transcription factor, CDX2, was common among all the miRs (Table S1).

TABLE S1
Distribution of transcription factor binding sites on the miR promoters

| miRNA | CDX2 | HNF4A | ZNF263 | TCF12 | Inil |
|---|---|---|---|---|---|
| miR-29a-Sp | 1 | 1 | 0 | 0 | 0 |
| miR-29b-3p | 1 | 1 | 0 | 1 | 0 |
| miR-96-5p | 1 | 1 | 1 | 1 | 1 |
| miR-139-5p | 1 | 1 | 1 | 0 | 0 |
| miR-182-5p | 1 | 1 | 1 | 0 | 1 |
| miR-183-5p | 1 | 1 | 1 | 1 | 1 |
| miR-335-3p | 1 | 1 | 1 | 1 | 1 |
| miR-378a-3p | 1 | 1 | 1 | 1 | 1 |
| miR-486-3p | 1 | 0 | 1 | 0 | 0 |
| miR-486-Sp | 1 | 0 | 1 | 0 | 0 |
| miR-561-5p | 1 | 1 | 1 | 1 | 1 |
| miR-584-5p | 1 | 1 | 1 | 1 | 1 |
| miR-625-3p | 1 | 1 | 0 | 0 | 0 |
| miR-1255a | 1 | 1 | 1 | 1 | 1 |
| miR-3934-5p | 1 | 1 | 0 | 0 | 1 |
| miR-6723-5p | 0 | 0 | 0 | 0 | 0 |

“1” indicates the presence of one or more transcription factor binding site while “0” indicates the lack of any binding site on the promoter of the indicated miR.

The inventors hypothesized that CDX2 (known to be expressed in colon) maybe controlling the expression of all the DDSMs. CDX2 expression was found to respond to the extent of DNA damage within the cells generated by HU treatment or IR exposure (FIG. 3A, 3B, S3A, S3B). EMSAs demonstrated that CDX2 bound to the promoter sequences of three tested DDSMs. Super-shifts were observed when an anti-CDX2 antibody was used in the EMSA reactions (FIG. 3C). The binding of CDX2 to the miR promoters was lost in presence of cold competitor (FIG. 3D) or when a mutant oligo in which CDX2 binding site was destroyed was used as the substrate (Figure S3C). Using immunoprecipitated (IPed) CDX2, it was shown that the binding of CDX2 to the miR promoters was enhanced 2hrs post-IR (FIG. 3E). The ability of CDX2 to transactivate the DDSMs in a dose-dependent manner was demonstrated for the wildtype protein and not for mini CDX2 lacking its transactivation domain (FIG. 3F, S3D). Mutating the CDX2 binding site on the miR promoters ablated the transactivation property of CDX2 (FIG. 3G). As complementary approach, the inventors identified three arginine residues in CDX2 (R190, R238 and R242) which when mutated to alanine abolished its ability to bind to the miR promoters. This was demonstrated using EMSAs (FIG. 3H) and luciferase assays (FIG. 3I). Indeed, expression of the DNA binding mutants of CDX2 showed substantial decrease in DDSM levels when compared to wildtype CDX2 (FIG. 3J). Correspondingly, ablation of CDX2 by siRNA decreased the levels of primary, precursor and mature miRs within the cells (FIG. 3K, S3E). Altogether these evidence prove that CDX2 is the common transcription factor upregulating the expression of the DDSMs.

The inventors next hypothesized that regulating the levels of CDX2 should also lead to regulation of the miR levels and thereby the neoplastic transformation process. First, the inventors used a HT29 based pair of doxycycline (Dox) inducible stable lines, namely TW6 (which expressed CDX2 after Dox treatment) and TG8 (which was the corresponding vector control). Both these lines also constitutively expressed GFP (FIG. 4A). Induction of CDX2 by Dox treatment in TW6 cells (FIG. 4A) along with concomitant exposure to HU led to an increase in the levels of the DDSMs (FIG. 4B), which culminated in enhanced wound healing (FIG. 4C) and colony formation (FIG. 4D) efficiencies. The stimulatory effect of CDX2 was also tested in another pair of cell lines based on HCT116 cells—namely HW2 (constitutively expressing Flag CDX2) and HC1 (the corresponding vector control) (Figure S4A). Overexpression of Flag CDX2 in HW2 cells led to the upregulation of the DDSMs (Figure S4B). Next, in vivo experiments were carried out using xenograft models where TG8 and TW6 cells were subcutaneously injected into NOD SCID mice, a subset of which were fed orally with Dox. Tumors obtained from TW6 cells expressing inducible CDX2 had the maximum tumor volume (FIG. 4E). Tumors were excised at the end point of the above experiment. Tumors obtained from TW6 cells showed increased levels of all the tested DDSMs (FIG. 4F), increased levels of the proliferation marker PCNA (FIG. 4G) and angiogenesis marker CD31 (FIG. 4H). The inventors also carried out in vivo subcutaneous xenograft studies using the HC1/HW2 cells. The sizes of tumors obtained from HW2 cells were larger (Figure S4C), they expressed higher levels of the DDSMs (Figure S4D), PCNA (Figure S4C) and CD31 (Figure S4D). Finally, the inventors wanted to determine whether expression of DNA damage inducible miRs caused increased dissemination of the cancer cells. Using the TG8/TW6 isogenic lines, the inventors carried out three experiments, namely a subcutaneous model (FIG. 4I), an intravenous model where cells were injected via tail vain (FIG. 4J) and an orthotopic model where the cells were implanted into the cecal wall of the mice (FIG. 4K). All the mice in each of the models were orally fed with Dox. At 21 days post-initiation of the experiments the mice were subjected to whole body imaging and GFP fluorescence tracked. In all the three experimental systems TW6 cells expressing CDX2 showed enhanced in vivo dissemination to distal organs (FIG. 4I-4K).

### Example 4: BRCA1 is a Target of the DDSMs

Having established how the DDSMs are regulated in the colon, the inventors wanted to decipher how these miRs function. For that the inventors determined the putative targets of the DDSMs. Using MiRanda, we found 2266 common targets for the eight DDSMs (Figure S5A, Table S2).

TABLE S2
Names of the genes targeted by all the miR promoters

| | | | | |
|---|---|---|---|---|
| AACS | ADAMTS16 | AKAP2 | ANKRD20B | ARHGAP29 |
| AAK1 | ADAMTS19 | AKAP6 | ANKRD26 | ARHGAP31 |
| ABAT | ADAMTS5 | AKAP8 | ANKRD36 | ARHGAP32 |
| ABCA11P | ADAMTSL3 | AKNA | ANKRD42 | ARHGEF12 |
| ABCA13 | ADARBI | AKR7L | ANKRD49 | ARHGEF15 |
| ABCA17P | ADAT2 | ALAD | ANKRD52 | ARHGEF37 |
| ABCB5 | ADCY1 | ALCAM | ANKRD6 | ARHGEF7 |
| ABCC12 | ADD3 | ALDHIL2 | ANKS1B | ARID2 |
| ABCC5 | ADH5 | ALDH5A1 | ANKS6 | ARID3B |
| ABCD2 | ADIPOQ | ALG10B | ANO6 | ARID4B |
| ABHD2 | ADNP2 | ALG11 | ANP32E | ARIH1 |
| ABHD4 | ADRBK2 | ALG6 | ANTXR1 | ARIH2 |
| ABI3BP | AFF1 | ALPK1 | ANTXR2 | ARL1 |
| ABL2 | AFF3 | ALPK3 | ANXA4 | ARL10 |
| ABLIM3 | AFF4 | ALS2 | APIS3 | ARL11 |
| ABT1 | AGAP9 | ALS2CR11 | AP2A2 | ARL13B |
| ACACB | AGFG1 | ALS2CR8 | AP3M2 | ARL17A |
| ACADSB | AGFG2 | AMACR | APBA1 | ARL4A |
| ACAP2 | AGK | AMICA1 | APBB2 | ARMC8 |
| ACCN2 | AGPAT4 | AMOTLI | APOL6 | ARMC9 |
| ACER3 | AGPAT5 | ANGPT2 | APOLD1 | ARMCX4 |
| ACO1 | AGPHD1 | ANK1 | APPBP2 | ARRDC4 |
| ACP6 | AGPS | ANKAR | APPL1 | ARSB |
| ACPL2 | AGXT2 | ANKFY1 | AQP4 | ARSK |
| ACSM2A | AHCYL1 | ANKIB1 | ARAP2 | ART3 |
| ACVR1C | AIG1 | ANKRD11 | ARHGAP19 | ASB7 |
| ACVR2B | AIPL1 | ANKRD12 | ARHGAP20 | ASPH |
| ADAM28 | AKAP12 | ANKRD13C | ARHGAP24 | ASTN1 |
| ATF3 | ATXN3 | BMP3 | C14orf119 | CIQTNF7 |
| ATF7 | ATXN7L1 | BMP8A | C14orf145 | C2 |
| ATF7IP | ATXN7L3B | BMPR2 | C14orf147 | C20orf11 |
| ATG3 | B3GALTL | BMS1 | C15orf27 | C20orf194 |
| ATG4A | B4GALT6 | BNC2 | C16orf54 | C20orf4 |
| ATM | BAALC | BRCA1 | C16orf57 | C21orf34 |
| ATP10B | BACHI | BRCC3 | C16orf70 | C21orf62 |
| ATP11C | BAG1 | BRD3 | C16orf72 | C21orf91 |
| ATP1A4 | BBS1 | BRWD1 | C17orf104 | C21orf99 |
| ATP1B1 | BBS2 | BTBD1 | C17orf108 | C2orf60 |
| ATP1B4 | BBS9 | BTN2A2 | C17orf51 | C2orf63 |
| ATP2B1 | BBX | BTRC | C17orf68 | C2orf68 |
| ATP2B2 | BCAP29 | BZRAP1 | C17orf99 | C2orf69 |
| ATP2B4 | BCKDK | C10orf25 | C18orf1 | C2orf71 |
| ATP5G3 | BCL11A | C10orf44 | C18orf25 | C2orf86 |
| ATP6VIA | BCL2L11 | C10orf72 | C19orf50 | C3orf52 |
| ATP7A | BCL2L14 | C11orf41 | Clorf112 | C3orf59 |
| ATP7B | BCL2L15 | C11orf45 | Clorf115 | C3orf63 |
| ATP8A1 | BDH1 | C11orf57 | Clorf161 | C3orf72 |
| ATP8A2 | BDKRB2 | C11orf87 | Clorf21 | C4orf10 |
| ATP8B4 | BDNF | C12orf29 | Clorf226 | C4orf12 |
| ATP9B | BEND4 | C12orf5 | Clorf43 | C4orf46 |
| ATPAF1 | BEND7 | C12orf50 | Clorf52 | C5orf22 |
| ATR | BETIL | C12orf55 | Clorf69 | C5orf24 |
| ATRN | BICD1 | C12orf65 | Clorf77 | C5orf41 |
| ATRNL1 | BICD2 | C13orf1 | Clorf87 | C5orf47 |
| ATXN1 | BLK | C13orf31 | Clorf9 | C5orf51 |
| ATXN2 | BMP2K | C14orf101 | C1QTNF6 | C6orf142 |
| C6orf164 | CACYBP | CCDC109A | CD226 | CEP350 |
| C6orf167 | CADM1 | CCDC117 | CD302 | CEP57 |
| C6orf168 | CADM2 | CCDC125 | CD33 | CEP78 |
| C6orf174 | CALCB | CCDC132 | CD36 | CFL2 |
| C6orf201 | CALD1 | CCDC144NL | CD44 | CFLAR |
| C6orf222 | CALN1 | CCDC149 | CD55 | CGGBP1 |
| C6orf89 | CAMTA1 | CCDC152 | CD59 | CHCHD1 |
| C7orf42 | CAND1 | CCDC30 | CD69 | CHD2 |
| C7orf46 | CANX | CCDC40 | CD96 | CHEK1 |
| C7orf54 | CAPN7 | CCDC50 | CDADC1 | CHL1 |
| C7orf60 | CAPRINI | CCDC52 | CDC27 | CHM |
| C7orf70 | CAPZA2 | CCDC68 | CDHR3 | CHML |
| C8orf12 | CARD8 | CCDC7 | CDK1 | CHORDC1 |
| C8orf79 | CASC4 | CCDC73 | CDK13 | CHPT1 |
| C9orf102 | CASP8 | CCDC82 | CDK16 | CHRM2 |
| C9orf109 | CATSPER2 | CCDC88A | CDK6 | CHST3 |
| C9orf110 | CATSPER4 | CCDC91 | CDKAL1 | CHST9 |
| C9orf170 | CAV2 | CCDC93 | CDKN2BAS | CLCN4 |
| C9orf3 | CBFA2T2 | CCNC | CDS1 | CLDN12 |
| C9orf30 | CBLL1 | CCND1 | CEACAM6 | CLDN2 |
| C9orf45 | CBR4 | CCND2 | CEACAM7 | CLEC12B |
| C9orf47 | CBWD5 | CCNDBP1 | CECR1 | CLEC2D |
| C9orf68 | CBX1 | CCNH | CELF2 | CLEC7A |
| C9orf85 | CBX3 | CCNT2 | CELSR1 | CLIC5 |
| CA5B | CBX5 | CCNY | CENPN | CLIC6 |
| CACNA1C | CBX6 | CCNYL1 | CENPW | CLIP4 |
| CACNA1E | CCAR1 | CCR1 | CEP135 | CLLU1 |
| CACNB4 | CCBE1 | CCT6P1 | CEP192 | CLMN |
| CLTC | CPEB3 | CXorf22 | DCX | DKFZp686024166 |
| CMAH | CPM | CXorf36 | DDAH1 | DKFZP781G0119 |
| CMKLR1 | CPPED1 | CXorf41 | DDB2 | DKK2 |
| CMTM1 | CPZ | CYB561D1 | DDHD1 | DLG2 |
| CNBP | CR1 | CYB5B | DDHD2 | DLGAP2 |
| CNOT6 | CRB1 | CYB5RL | DDX10 | DMD |
| CNOT6L | CREB1 | CYCS | DDX3X | DMTF1 |
| CNP | CREBL2 | CYFIP2 | DDX60 | DMXL1 |
| CNST | CREG2 | CYLD | DDX60L | DNAH14 |
| CNTLN | CREM | CYP1B1 | DENND3 | DNAH5 |
| CNTN3 | CRTC3 | CYP20A1 | DENND4C | DNAJB14 |
| CNTNAP2 | CS | CYP27C1 | DENND5B | DNAJC10 |
| COG5 | CSMD3 | CYP2B7P1 | DERL1 | DNAJC16 |
| COL19A1 | CSNKIG1 | CYP2U1 | DFFB | DNAJC21 |
| COL29A1 | CSPP1 | CYP46A1 | DGCR14 | DNAJC24 |
| COL4A3 | CSRNP2 | CYP4V2 | DGCR5 | DNAJC3 |
| COL4A3BP | CTNNA1 | DAPP1 | DGKB | DNAJC5B |
| COL4A5 | CTNNB1 | DAZAP2 | DGKE | DNAJC6 |
| COL5A1 | CTNNBL1 | DBF4B | DHFRL1 | DNAL1 |
| COMMD9 | CTSB | DBT | DHRS4L2 | DNM2 |
| COPS7A | CTSO | DCAF10 | DHRSX | DNM3 |
| COPS7B | CTSS | DCAF16 | DHX33 | DOCK2 |
| COQ9 | CTTNBP2NL | DCAF17 | DIO2 | DOCK3 |
| CORIN | CUL3 | DCAF4L1 | DIP2B | DOCK5 |
| CORO2A | CUL5 | DCAF7 | DISC1 | DOCK8 |
| COX10 | CUX2 | DCLK1 | DIXDC1 | DOK6 |
| COX15 | CXCL9 | DCLK3 | DKFZP434L187 | DPPA4 |
| CPAMD8 | CXorf1 | DCP2 | DKFZp686D0853 | DPY19L3 |
| DSC3 | ELAVL2 | EXOC5 | FAM151B | FBXO42 |
| DSEL | ELP2 | EXOC6 | FAM155B | FBXO45 |
| DSG3 | EML1 | EXOC8 | FAM160B2 | FBXO48 |
| DST | EML6 | EYS | FAM167A | FBX07 |
| DUS4L | ENAH | F2R | FAM169B | FBX09 |
| DUSP28 | ENC1 | F2RL2 | FAM175B | FBXW11 |
| DUXAP10 | ENPP5 | FABP2 | FAM177A1 | FBXW2 |
| DYNC1LI2 | ENTPD1 | FAHD1 | FAM178A | FBXW8 |
| DYNLRB1 | EPB41L5 | FAIM | FAM182A | FCHSD2 |
| DYSF | EPHA3 | FAM100B | FAM190A | FCRL5 |
| DYT3 | EPHA7 | FAM102B | FAM190B | FECH |
| DZIP3 | EPM2AIP1 | FAM105B | FAM193A | FER |
| E2F5 | EPT1 | FAM115C | FAM198B | FERMT1 |
| EDA | ERAP2 | FAM116A | FAM20B | FERMT2 |
| EDEM1 | ERBB3 | FAM119A | FAM23A | FGB |
| EDEM3 | ERBB4 | FAM120A | FAM23B | FGD4 |
| EDN1 | ERC1 | FAM122B | FAM55C | FGF2 |
| EDNRB | ERGIC3 | FAM122C | FAM65B | FGF5 |
| EEA1 | ERLIN2 | FAM126A | FAM84B | FILIP1 |
| EFCAB1 | ERO1LB | FAM126B | FAM95B1 | FKBP5 |
| EFHC1 | ERP44 | FAM127C | FAS | FKTN |
| EFNA5 | ESCO2 | FAM129A | FBN2 | FLJ10038 |
| EFR3B | ESR1 | FAM129C | FBXL17 | FLJ10489 |
| EIF2C1 | ESRRG | FAM134A | FBXL18 | FLJ10661 |
| EIF2S2 | ETS1 | FAM134C | FBXO28 | FLJ11292 |
| EIF4E2 | ETV1 | FAM135A | FBX036 | FLJ21408 |
| EIF4E3 | ETV6 | FAM135B | FBXO40 | FLJ30307 |
| EIF4EBP2 | EVC | FAM13AOS | FBXO41 | FLJ39080 |
| FLJ39582 | FUNDC2 | GGPS1 | GPC6 | GTF2H5 |
| FLJ40288 | FUT4 | GHR | GPNMB | GTF2IRD2 |
| FLJ40330 | FUT9 | GIGYF2 | GPR12 | GTF3A |
| FLJ43315 | FYCO1 | GIT2 | GPR126 | GTPBP10 |
| FLJ43390 | FZD1 | GJA3 | GPR158 | GUCY1A3 |
| FLJ43950 | FZD4 | GJC1 | GPR21 | GULP1 |
| FLJ45244 | G6PC | GK5 | GPR26 | GXYLT1 |
| FLJ45340 | GAB1 | GKN2 | GPR64 | GYPC |
| FLJ46361 | GAB3 | GLCE | GPR89A | H2AFJ |
| FLRT3 | GABARAPL1 | GLIPR1 | GPR98 | H2AFV |
| FLT1 | GABRA4 | GLP1R | GPRIN3 | HAPLN1 |
| FMN1 | GABRB2 | GM2A | GPX8 | HAUS6 |
| FNTA | GABRG1 | GMFB | GRAMDIC | HBP1 |
| FOXK2 | GABRG2 | GNA13 | GRAMD3 | HBS1L |
| FOXN3 | GABRP | GNA01 | GREM1 | HCG22 |
| FOXO3 | GALNT10 | GNAS | GRIA1 | HCN1 |
| FOXP1 | GALNTL5 | GNB4 | GRIA3 | HDAC4 |
| FOXP2 | GANC | GNG12 | GRIA4 | HEATR7A |
| FRAS1 | GAPVD1 | GNG4 | GRID2 | HECTD2 |
| FREM2 | GATADI | GNG7 | GRIK3 | HELB |
| FRG1B | GATAD2B | GNL3L | GRIN2A | HELZ |
| FRMD4A | GCLM | GNPDA2 | GRIN2C | HERC4 |
| FRMD4B | GCNT1 | GNS | GRIN3A | HES2 |
| FRMD6 | GCNT2 | GOLGA3 | GRM6 | HEXA |
| FRMPD4 | GCOM1 | GOSR1 | GRM7 | HHLA1 |
| FRY | GDAP2 | GPAM | GRSF1 | HIBADH |
| FSTL4 | GEN1 | GPBP1 | GSK3B | HIF3A |
| FSTL5 | GFPT1 | GPC3 | GSTM3 | HINT3 |
| HIP1 | ICMT | INPP4A | KBTBD11 | KIAA0776 |
| HIPK2 | IDO2 | INPP5B | KBTBD2 | KIAA0831 |
| HIVEP1 | IDS | INSR | KBTBD6 | KIAA0892 |
| HLA-DOA | IFI44L | INTS6 | KBTBD8 | KIAA0895 |
| HLA-F | IFRD1 | INTS8 | KCNC1 | KIAA0907 |
| HLTF | IFT27 | IPCEF1 | KCND2 | KIAA1199 |
| HMGA2 | IGF1 | IPO8 | KCNE1 | KIAA1211 |
| HMGCLL1 | IGF2BP1 | IPO9 | KCNIP3 | KIAA1244 |
| HNRNPM | IGF2BP3 | IQCH | KCNK2 | KIAA1267 |
| HNRNPU | IGFBP5 | IQSEC1 | KCNMA1 | KIAA1598 |
| HOMEZ | IGFN1 | IRAK4 | KCNMB2 | KIAA1609 |
| HOOK1 | IKBIP | IREB2 | KCNN3 | KIAA1715 |
| HOOK3 | IKBKB | IRF1 | KCNU1 | KIAA1841 |
| HP1BP3 | IKZF2 | IRGQ | KCTD12 | KIAA2018 |
| HPGD | IL11RA | ISPD | KCTD20 | KIAA2022 |
| HPS1 | IL16 | ITPR2 | KDSR | KIF13B |
| HPS3 | IL17RD | ITSN1 | KGFLP1 | KIF1B |
| HRASLS5 | IL1RAP | IYD | KHNYN | KIF23 |
| HRH1 | ILIRL1 | JAKMIP3 | KIAA0125 | KIF3A |
| HRH2 | IL23R | JAM3 | KIAA0182 | KITLG |
| HS2ST1 | IL6R | JHDMID | KIAA0240 | KLF12 |
| HS6ST3 | IMPA1 | JOSD1 | KIAA0319L | KLF13 |
| HSP90AB2P | IMPAD1 | JPH2 | KIAA0355 | KLF3 |
| HSPA12A | INADL | JRK | KIAA0408 | KLF4 |
| HSPB11 | ING3 | KAL1 | KIAA0494 | KLHL18 |
| HSPC159 | INMT | KALRN | KIAA0513 | KLHL21 |
| HTR2C | INO80C | KANK1 | KIAA0652 | KLHL29 |
| HYDIN | INO80D | KAT2B | KIAA0664P3 | KLHL3 |
| KLHL32 | LNX2 | LOC253039 | LOC344595 | LOC84740 |
| KLHL6 | LOC100124692 | LOC255187 | LOC348120 | LOC90246 |
| KLHL8 | LOC100128025 | LOC256880 | LOC387647 | LOC96610 |
| KPNA1 | LOC100128098 | LOC283267 | LOC388692 | LONRF2 |
| KRT37 | LOC100129034 | LOC283314 | LOC389765 | LOX |
| KSR1 | LOC100129055 | LOC283508 | LOC400622 | LPGAT1 |
| KSR2 | LOC100129550 | LOC283914 | LOC440416 | LPP |
| KTELC1 | LOC100129826 | LOC284023 | LOC441204 | LPPR5 |
| LAMC1 | LOC100130354 | LOC284233 | LOC550112 | LRAT |
| LAMC2 | LOC100131190 | LOC284561 | LOC550113 | LRMP |
| LARP4 | LOC100132077 | LOC284577 | LOC642597 | LRP11 |
| LARP4B | LOC100132352 | LOC284900 | LOC643327 | LRP12 |
| LASS6 | LOC100132707 | LOC285026 | LOC643406 | LRRC2 |
| LCLAT1 | LOC100133029 | LOC285045 | LOC643763 | LRRC27 |
| LCP1 | LOC100133091 | LOC285194 | LOC645323 | LRRC37B2 |
| LEPR | LOC100270804 | LOC285540 | LOC646851 | LRRC55 |
| LEPRE1 | LOC100286844 | LOC285556 | LOC648691 | LRRC57 |
| LGR5 | LOC143188 | LOC285593 | LOC650623 | LRRC58 |
| LGSN | LOC145820 | LOC285954 | LOC727896 | LRRC59 |
| LHFPL2 | LOC145837 | LOC285965 | LOC728264 | LRRC7 |
| LHFPL3 | LOC148696 | LOC286135 | LOC728323 | LRRC9 |
| LHFPL4 | LOC149134 | LOC286367 | LOC728640 | LRRFIP1 |
| LHX9 | LOC149773 | LOC338579 | LOC728716 | LRRK2 |
| LIFR | LOC150577 | LOC338739 | LOC729723 | LRRTM2 |
| LIMD2 | LOC150622 | LOC339400 | LOC730101 | LRRTM3 |
| LIPG | LOC154822 | LOC339524 | LOC730668 | LUZP1 |
| LIX1 | LOC158435 | LOC339862 | LOC731789 | LYPD6 |
| LMNB1 | LOC221442 | LOC340113 | LOC80154 | LYRM7 |
| MACROD2 | MED13L | MIER1 | MTRR | NAV2 |
| MAGI1 | MEF2C | MINA | MTX3 | NBEA |
| MAGI3 | MEGF10 | MIPOL1 | MUC17 | NBLA00301 |
| MAMDC2 | MEGF6 | MKNK2 | MUC3A | NCAM1 |
| MAML3 | MEGF9 | MLEC | MXD1 | NCOA1 |
| MAOA | MEI1 | MMAA | MXI1 | NCOA7 |
| MAP2 | MEIS1 | MMP16 | MXRA7 | NCRNA00103 |
| MAP2K4 | MEOX2 | MN1 | MYEF2 | NCRNA00183 |
| MAP2K6 | MESDC2 | MOBKLIA | MYLK3 | NCRNA00200 |
| MAP3K4 | METAP1 | MOCS2 | MYNN | NCRNA00222 |
| MAP3K5 | METT5D1 | MPPED2 | MYO10 | NDFIP2 |
| MAP3K9 | METTL9 | MPST | MYO18A | NDST3 |
| MAP9 | MEX3A | MPZL1 | MYO1B | NDUFA5 |
| MAPK1 | MEX3C | MPZL2 | MYO3B | NECAB1 |
| MAPK4 | MFAP3L | MRO | MYO6 | NEDD4L |
| MAPKSP1 | MFSD4 | MRPL19 | MY09A | NEGR1 |
| MASP1 | MGAT4A | MRPS11 | MYOZ3 | NEK11 |
| MAST2 | MGC11082 | MRS2 | N4BP2L1 | NEURLIB |
| MBD6 | MGC23284 | MSH2 | NAA16 | NEURODI |
| MBP | MGC34034 | MSI2 | NAA30 | NEUROD4 |
| MCC | MGC57346 | MSR1 | NAAA | NEUROG2 |
| MCFD2 | MIA3 | MSRB2 | NAIP | NF1 |
| MCM5 | MIAT | MTAP | NAMPT | NFASC |
| MCPH1 | MIB1 | MTHFD2L | NANOS1 | NFAT5 |
| MCTS1 | MICA | MTMR9 | NANP | NFIA |
| MDFIC | MICAL2 | MTPAP | NARF | NFX1 |
| MDM2 | MICAL3 | MTR | NARG2 | NFYA |
| MECP2 | MID2 | MTRFIL | NAVI | NHLRC2 |
| NHS | NUDT16P1 | PACS1 | PCDH19 | PDXK |
| NIPAL3 | NUFIP1 | PACSIN1 | PCDH7 | PDZD2 |
| NISCH | NUFIP2 | PAFAHIBI | PCDH9 | PEF1 |
| NKAIN2 | NUMA1 | PAG1 | PCDHA13 | PELI2 |
| NLGN1 | NUMB | PAIP2B | PCDHA4 | PERP |
| NLGN4Y | NUPL2 | PALM2 | PCDHA5 | PEX14 |
| NLRP3 | NXF4 | PALM2-AKAP2 | PCGF3 | PEX5 |
| NMD3 | OAS2 | PAN3 | PCLO | PFKFB2 |
| NME7 | OAS3 | PAPD5 | PCNX | PFN2 |
| NOD2 | ODZ1 | PAPOLG | PCSK5 | PGAP1 |
| NOM1 | OLA1 | PAPPA | PCYOX1 | PGM2L1 |
| NOP56 | ONECUT2 | PAPPA2 | PCYT1B | PGR |
| NPAS3 | OPA1 | PAQR8 | PDE10A | PHACTRI |
| NPLOC4 | OPCML | PARP14 | PDE3B | PHACTR2 |
| NPTX1 | ORAI2 | PARP9 | PDE4D | PHACTR4 |
| NR1D2 | ORC3L | PARTI | PDE7A | PHC3 |
| NR2C1 | OSBPL11 | PATZ1 | PDE7B | PHF15 |
| NR2C2 | OSBPL3 | PAX6 | PDGFA | PHF17 |
| NR3C2 | OTUD3 | PAX7 | PDGFRA | PHF20L1 |
| NRG1 | OTUD4 | PAX8 | PDHA1 | PHF21A |
| NRXN1 | OTUD7B | PBX1 | PDK1 | PHF3 |
| NRXN3 | OVOS | PBX3 | PDLIM2 | PHF6 |
| NSL1 | OXA1L | PCA3 | PDLIM5 | PHIP |
| NSUN4 | OXNAD1 | PCBD2 | PDPR | PHLDB1 |
| NTRK2 | OXTR | PCBP2 | PDRG1 | PHLDB2 |
| NUB1 | P2RX5 | PCDH11X | PDS5A | PHLPP2 |
| NUBPL | P704P | PCDH15 | PDS5B | PHTF2 |
| NUDT16 | PAAF1 | PCDH17 | PDSS2 | PI15 |
| PIGK | POF1B | PPP2CA | PSTPIP2 | RAB27A |
| PIK3AP1 | POGK | PPP2R5E | PTAR1 | RAB31 |
| PIK3C2G | POLI | PPP3CA | PTBP2 | RAB3IP |
| PIK3C3 | POLK | PPP6C | PTCD3 | RAB8B |
| PIK3R1 | POLR1D | PRDM10 | PTEN | RABEP1 |
| PIK3R3 | POLR3F | PRDM2 | PTGDS | RABGAP1 |
| PIK3R5 | POLR3G | PRDM6 | PTGER3 | RABGAPIL |
| PIP5K1B | POM121 | PRELID2 | PTGFR | RAD23B |
| PIWIL4 | POM121C | PREPL | PTP4A1 | RAD51L1 |
| PKHD1 | POM121L8P | PRKAA2 | PTPDC1 | RALBP1 |
| PKN2 | POM121L9P | PRKAR1A | PTPLAD2 | RALGAPB |
| PLA2G12A | POU3F2 | PRKCA | PTPN3 | RALGPS2 |
| PLAC8 | PPARD | PRKCE | PTPRB | RANBP10 |
| PLAG1 | PPCDC | PRKD3 | PTPRC | RANBP17 |
| PLAGL2 | PPFIA1 | PRKX | PTPRD | RANBP6 |
| PLB1 | PPHLN1 | PRKY | PTPRE | RAPIGDS1 |
| PLCB1 | PPIE | PRLR | PTPRG | RAP2A |
| PLCB4 | PPIF | PRMT8 | PTPRT | RAPGEF3 |
| PLCXD2 | PPIP5K2 | PROM2 | PTTG1IP | RAPGEF4 |
| PLCXD3 | PPM1A | PRPF38A | PURB | RAPH1 |
| PLEKHG2 | PPM1D | PRPF40A | PURG | RASAL2 |
| PLEKHH1 | PPM1H | PRPF4B | PVT1 | RASGRF1 |
| PLEKHH2 | PPM1K | PRTG | PXMP4 | RASGRF2 |
| PLEKHM3 | PPM1L | PSD3 | QKI | RB1 |
| PLIN4 | PPP1R12B | PSEN1 | QSER1 | RBBP9 |
| PLSCR1 | PPPIRIC | PSEN2 | QTRTD1 | RBM12 |
| PLXNA4 | PPP1R2P1 | PSMA2 | RAB12 | RBM15B |
| PML | PPP1R3B | PSMC6 | RAB22A | RBM33 |
| RBM46 | RNF114 | RUNX1 | SEC23IP | SH3RF2 |
| RBM9 | RNF125 | RUNX1T1 | SEC62 | SH3TC2 |
| RBMS1 | RNF144A | RUNX2 | SEC63 | SHANK2 |
| RBMS2 | RNF144B | RYR2 | SEL1L | SHB |
| RBMXL1 | RNF157 | S100A7A | SELT | SHE |
| RBPJ | RNF169 | S100PBP | SEMA3D | SHISA7 |
| RCOR1 | RNF17 | SAMD12 | SEPSECS | SHPRH |
| RDX | RNF170 | SAMD4A | SERBP1 | SHROOM4 |
| RECK | RNF180 | SARMI | SERPINE1 | SIDT1 |
| REPS2 | RNF8 | SASH1 | SESTD1 | SIN3A |
| REXO1L2P | RNMT | SBF2 | SETD1B | SIX4 |
| RGMB | ROD1 | SBK1 | SF3A1 | SKP1 |
| RGNEF | RORA | SC5DL | SF3B3 | SLAIN2 |
| RGPD4 | RPAP2 | SCAI | SFRS1 | SLAMF7 |
| RGS6 | RPL13AP17 | SCAMP1 | SFRS11 | SLC11A2 |
| RGS7BP | RPL28 | SCAMP5 | SFRS12IP1 | SLC12A2 |
| RHOBTB3 | RPP14 | SCHIP1 | SFRS13A | SLC1A2 |
| RIC3 | RPP30 | SCML4 | SFRS18 | SLC22A10 |
| RIC8B | RPRD1A | SCN11A | SFRS2 | SLC22A15 |
| RIF1 | RPRD2 | SCN2A | SFRS5 | SLC22A8 |
| RIMKLA | RPS6KA3 | SCN3B | SFXN5 | SLC22A9 |
| RIMKLB | RPS7 | SCN5A | SGCD | SLC25A22 |
| RIMS1 | RREB1 | SCN9A | SGTB | SLC25A23 |
| RIMS2 | RRP15 | SCRN1 | SH3BGRL2 | SLC25A26 |
| RLIM | RRP1B | SDC3 | SH3BP1 | SLC25A36 |
| RMND5A | RSAD2 | SEC11C | SH3BP4 | SLC26A2 |
| RMST | RTEL1 | SEC14L2 | SH3GLB1 | SLC26A7 |
| RND3 | RTKN2 | SEC22A | SH3PXD2A | SLC2A10 |
| SLC2A12 | SMAD2 | SPAG16 | ST3GAL1 | SUZ12P |
| SLC2A13 | SMAD4 | SPAG9 | ST6GAL2 | SVIL |
| SLC2A4 | SMARCAL1 | SPATA20 | ST8SIA1 | SYNCRIP |
| SLC30A4 | SMARCE1 | SPATA5 | ST8SIA3 | SYNE1 |
| SLC30A7 | SMC1A | SPATS2 | ST8SIA4 | SYNJ2 |
| SLC33A1 | SMCHD1 | SPCS3 | STAG3L3 | SYNJ2BP |
| SLC35A1 | SMCR7L | SPEF2 | STAG3L4 | SYNPO2 |
| SLC35E2 | SMCR8 | SPG20 | STAMBPL1 | SYNPR |
| SLC35F1 | SMURF1 | SPIN1 | STARD13 | SYNRG |
| SLC36A2 | SNAPC3 | SPIRE1 | STAT1 | SYT11 |
| SLC38A1 | SNCAIP | SPN | STC1 | SYT13 |
| SLC38A2 | SNORD108 | SPOCK1 | STEAP4 | SYT14 |
| SLC46A1 | SNRNP48 | SPOCK3 | STK3 | SYT7 |
| SLC4A10 | SNRPN | SPON2 | STK38L | SYT9 |
| SLC4A5 | SNTB1 | SPOPL | STK4 | SYTL4 |
| SLC4A7 | SNTB2 | SPRED2 | STON1 | TAB2 |
| SLC4A8 | SNX1 | SPRY3 | STOX2 | TACC1 |
| SLC5A12 | SNX13 | SPRY4 | STX6 | TAF15 |
| SLC5A3 | SOBP | SPTLC2 | STXBP4 | TAL1 |
| SLC6A15 | SOCS4 | SPTY2D1 | STXBP5 | TANC1 |
| SLC6A17 | SOCS6 | SR140 | STXBP5L | TANC2 |
| SLC6A20 | SON | SRGAP3 | STYX | TAOK1 |
| SLC7A1 | SORBS1 | SRI | SUB1 | TAOK3 |
| SLC7A14 | SOS1 | SSFA2 | SUDS3 | TAP2 |
| SLC7A5 | SOX13 | SSH2 | SUFU | TAX1BP3 |
| SLCO4C1 | SOX6 | SSR1 | SULF1 | TBC1D12 |
| SLIT1 | SP100 | SSR2 | SURF4 | TBC1D15 |
| SLITRK6 | SP140 | SSX2IP | SUSD1 | TBC1D20 |
| TBC1D26 | THRB | TMEM189- | TNRC6C | TSNAX |
| | | UBE2V1 | | |
| TBC1D8B | THSD4 | TMEM189- | TOP1 | TSNAX-DISC1 |
| | | UBE2V1 | | |
| TBL1XR1 | THUMPD1 | TMEM194A | TOX4 | TSPAN11 |
| TBRG1 | TIAM1 | TMEM20 | TP63 | TSPYL2 |
| TCAM1P | TIGD7 | TMEM232 | TPM3 | TSPYL4 |
| TCEANC | TIMP3 | TMEM26 | TPRG1 | TTC14 |
| TCF12 | TINAG | TMEM30A | TPTE2P1 | TTC18 |
| TCF21 | TKTL2 | TMEM47 | TRA2B | TTC19 |
| TCF4 | TLE1 | TMEM57 | TRAF3IP1 | TTC23 |
| TCP10 | TLL2 | TMEM64 | TRAM2 | TTC23L |
| TCP11L2 | TLN2 | TMEM87A | TRAPPC10 | TTC39A |
| TCTN1 | TMC8 | TMEM9 | TRDMT1 | TTL |
| TDP1 | TMCC3 | TMF1 | TRIM14 | TTTY4B |
| TECPR2 | TMCO1 | TMOD2 | TRIM2 | TUFT1 |
| TESK2 | TMED10P1 | TMPO | TRIM25 | TUG1 |
| TET2 | TMED2 | TMPRSS11BNL | TRIM27 | TXLNB |
| TET3 | TMEFF2 | TMTC1 | TRIM33 | TXNL1 |
| TEX9 | TMEM108 | TMTC3 | TRIM4 | TXNRD1 |
| TFCP2L1 | TMEM117 | TMX1 | TRIM44 | TYW1 |
| TFEC | TMEM127 | TNFSF4 | TRIM66 | TYW3 |
| TFPI | TMEM128 | TNFSF8 | TRIM67 | UACA |
| TGFB2 | TMEM132B | TNK1 | TRIM9 | UBA6 |
| TGFBR1 | TMEM135 | TNKS | TRPC5 | UBASH3B |
| TGM2 | TMEM154 | TNKS2 | TRPM8 | UBE2CBP |
| TGM4 | TMEM167B | TNNI3K | TRPS1 | UBE2G2 |
| THADA | TMEM17 | TNPO1 | TSG1 | UBE2V1 |
| THAP2 | TMEM181 | TNRC6A | TSGA14 | UBE2V2 |
| THAP6 | TMEM183A | TNRC6B | TSIX | UBE2W |
| UBE3B | VAPA | WFDC13 | ZBTB49 | ZMYND17 |
| UBE3C | VAT1L | WHAMML2 | ZBTB8A | ZNF10 |
| UBE4B | VCL | WHSC1 | ZC3H6 | ZNF167 |
| UBIAD1 | VEZT | WHSCIL1 | ZC3H7B | ZNF182 |
| UBR2 | VGLL3 | WIPF1 | ZCCHC11 | ZNF189 |
| UBR3 | VPS13A | WIPF2 | ZCCHC14 | ZNF192 |
| UBTD2 | VPS13D | WNT4 | ZCCHC3 | ZNF208 |
| UBXN2B | VPS35 | WRB | ZDHHC14 | ZNF215 |
| UFD1L | VPS37B | WSB1 | ZDHHC15 | ZNF226 |
| UGGT1 | VPS45 | WSCD2 | ZDHHC17 | ZNF230 |
| UGT8 | VSTM2A | WWC2 | ZDHHC2 | ZNF236 |
| UHRF1BP1 | VWA3A | XAF1 | ZDHHC22 | ZNF24 |
| UHRF2 | VWA3B | XIAP | ZDHHC23 | ZNF250 |
| UMODL1 | VWA5B1 | XK | ZDHHC3 | ZNF252 |
| UNC119B | WASF3 | XPO4 | ZEB1 | ZNF26 |
| UNC80 | WASL | XPR1 | ZEB2 | ZNF264 |
| UPRT | WDFY2 | XRRA1 | ZFAND3 | ZNF276 |
| UQCC | WDFY3 | YAP1 | ZFAND5 | ZNF280D |
| USP15 | WDR20 | YES1 | ZFHX3 | ZNF284 |
| USP37 | WDR27 | YKT6 | ZFP106 | ZNF286A |
| USP45 | WDR35 | YME1L1 | ZFP3 | ZNF286B |
| USP48 | WDR36 | YPEL1 | ZFP36L2 | ZNF295 |
| USP6 | WDR41 | ZAK | ZHX1 | ZNF320 |
| USP6NL | WDR48 | ZAN | ZHX3 | ZNF333 |
| USP9X | WDR67 | ZBTB34 | ZIC3 | ZNF337 |
| USP9Y | WDR69 | ZBTB40 | ZKSCAN5 | ZNF33A |
| USPL1 | WDR72 | ZBTB41 | ZMYM1 | ZNF33B |
| VANGL1 | WDTC1 | ZBTB44 | ZMYM6 | ZNF362 |
| ZNF365 | ZNF611 | ZSCAN29 | SOCS6 | PPP2R5C |
| ZNF37A | ZNF614 | ZXDC | DUSP10 | PEX19 |
| ZNF384 | ZNF618 | ZYG11B | STC1 | L3MBTL3 |
| ZNF3970S | ZNF629 | ZZEF1 | NRG1 | BAZIB |
| ZNF436 | ZNF638 | ZZZ3 | RCN2 | IRS1 |
| ZNF445 | ZNF639 | ABAT | ANKMY2 | PKD2 |
| ZNF451 | ZNF652 | AKAP12 | LRP6 | TMEM184C |
| ZNF460 | ZNF655 | PIGX | IPPK | GCLM |
| ZNF470 | ZNF662 | PFN2 | C11orf63 | KCNK2 |
| ZNF480 | ZNF7 | PTPN4 | RHPN2 | SREK1IP1 |
| ZNF490 | ZNF706 | REV1 | MACROD2 | C11orf75 |
| ZNF493 | ZNF714 | ITGB1 | GTF2H1 | ROBO2 |
| ZNF496 | ZNF716 | KCNK10 | PPP2CA | SPRY2 |
| ZNF498 | ZNF720 | C20orf177 | FAM175B | CELF1 |
| ZNF507 | ZNF738 | FRMD6 | NTN4 | EEF2 |
| ZNF514 | ZNF740 | ABCB10 | GNG5 | ARPP19 |
| ZNF518A | ZNF761 | SLAIN1 | TMPO | THAP7 |
| ZNF518B | ZNF765 | ATP2B4 | NPAS3 | PHF15 |
| ZNF525 | ZNF780B | TTC7B | PDCD4 | STYX |
| ZNF527 | ZNF806 | C2orf44 | SLC25A15 | FCHO2 |
| ZNF529 | ZNF808 | KIAA0284 | RPS6KA3 | ATP2C1 |
| ZNF542 | ZNF81 | CSMD1 | SELIL | KIAA0101 |
| ZNF549 | ZNF813 | MAL2 | KIF2A | EZR |
| ZNF568 | ZNF814 | CPM | BTG1 | ARHGAP21 |
| ZNF578 | ZNF839 | TCF7L2 | PPP2CB | RBMS1 |
| ZNF587 | ZNF862 | PSEN2 | IDH2 | VSX2 |
| ZNF606 | ZNF880 | SACS | ANKRD50 | TOMM70A |
| ZNF609 | ZPLD1 | NEFL | TCF12 | MRC2 |
| C16orf72 | | | | |
| YAF2 | | | | |
| CTDSPL | | | | |
| QKI | | | | |

Using Reactome, KEGG and GSEA the inventors determined that the DDSMs regulated many vital cellular processes like cell-cell communications, programmed cell death, DNA replication and importantly DNA repair, gene expression and signal transduction (data not shown). The inventors chose four key genes involved in DDR namely BRCA1, ATM, Chk1, RNF8 for further experimentation. Decrease of the high levels of endogenous DDSMs by treatment with specific miR inhibitors led to increase in the transcript levels of BRCA1 (FIG. 5A, S5B), ATM (FIG. 5B), Chk1 (FIG. 5C), RNF8 (FIG. 5D) in both GM03509 GFP Clone 100 (FIG. 5A-5D) and HCT116 BLM−/− (Figure S5B) cells. Reciprocally, enhancement of the levels of DDSMs by treatment with specific mimics led to the decrease in the transcript levels of BRCA1 (FIG. 5E, S5C), ATM (FIG. 5F), Chk1 (FIG. 5G), RNF8 (FIG. 5H) in both GM03509 GFP-BLM Clone 4.3.4 (FIG. 5E-5H) and HCT116 WT (Figure S5C) cells. The effect of the miR inhibitors and mimics on the four targets at the RNA level was phenocopied at the protein levels in both cell types tested (FIG. 5I, 5J, S5D, S5E).

The inventors chose BRCA1 for mechanistic studies as BRCA1 mutation carriers has been reported to confer increased risk to colon cancer which has been supported by evidence obtained from meta-analysis (Oh, McBride et al., 2018). The levels of BRCA1 were upregulated when the miRs were downregulated in diverse experimental systems—including transient transfection systems in multiple cell types (FIG. 5I, S5D), stable lines (Figure S2C, S2D) and in the tumors obtained in two xenograft models where expression of DDSMs are inhibited (FIG. 2H, 2K). Reciprocally, the inventors also observed that the levels of BRCA1 was downregulated when the miR levels were enhanced in multiple experimental systems—cells exposed to a gradient of different types of DNA damages (Figure S5F, S5G), high CDX2 expression levels (Figure S5H, S5I), transient transfection systems by treatment with miR mimics (FIG. 5J, S5E) and in tumors obtained from two xenograft models where the levels of the miRs were increased by inducible or constitutive enhancement of CDX2 levels (FIG. 4G, S4E). Interestingly, lack of CDX2 DNA binding activity resulted in upregulation of BRCA1 transcription, thereby again demonstrating the direct CDX2-DDSM-BRCA1 linkage (Figure S3F). Analysis of the 3′ UTR of BRCA1 revealed binding sites of the DDSMs (Figure S5J). The inventors used a luciferase reporter assay using either wildtype (WT) or mutant (MT) 3′BRCA1 UTR to demonstrate that overexpression of three of the DDSMs led to the ablation of luciferase activity only when WT BRCA1 3′UTR was used (FIG. 5K). The inventors hypothesized that if DDSMs act through BRCA1, increasing or decreasing BRCA1 levels should phenocopy the effects seen by modulating the levels of the miRs in cellular invasion assays (FIG. 2D, 2E). Indeed, ablation of BRCA1, verified at both RNA and protein levels (Figure S6A, S6B) led to increase in the level endogenous DNA damage (Figure S6C), SCEs (Figure S6D) and invasion (Figure S6E). Conversely, overexpression of BRCA1 (Figure S6F), decreased the DNA damage levels (Figure S6G), SCEs (Figure S6H) and invasion (Figure S6I).

### Example 5: BLM Represses CDX2 Expression

Having demonstrated that CDX2 is the common transcription factor which upregulates the expression of the DDSMs (FIG. 3, 4, S3, S4), the inventors next wanted to determine the upstream regulatory factor which keeps the DDSMs in “off” state under normal conditions. The inventors hypothesized that BLM itself may negatively regulate CDX2 expression as the levels of CDX2 were elevated in HCT116 BLM−/− cells (FIG. 6A, S7A). Further, overexpression of BLM reduced the transcript level of CDX2 (FIG. 6B). The inventors next wanted to determine whether BLM is recruited to the promoter of CDX2. On analysis of the 5 kb upstream to the CDX2 promoter TSS, the inventors identified potential binding sites for transcriptional repressors—MAD, AP3β, SMAD3, AP2β (Figure S7B). Additionally, the inventors also identified binding sites for transcriptional activator, E2F1, on the CDX2 promoter. Using ChIP, the inventors found that BLM was specifically recruited to the putative binding sites of all the tested transcriptional repressors, but not to the E2F1 binding site (FIG. 6C). Two transcriptional repressors, SMAD3 and AP2β, were themselves recruited to their cognate binding sites in a BLM dependent manner (FIG. 6D, 6E).

Transcriptional repression is controlled by two major remodeling co-repressor protein complexes—NuRD and Sin3. These two repressor complexes have specific subunits and also share common subunits (Baymaz, Karemaker et al., 2015). LC MS/MS analysis of immunoprecipitated BLM indicated its interaction with both Sin3b and CHD4, the core ATPase subunits of the two complexes. The peptide sequences found associated with BLM immunoprecipitates were SQSIDTPGVIR (for Sin3b) and APEPTPQQVAQQQ (for CHD4). BLM or Sin3b immunoprecipitations further revealed that BLM interacted with Sin3b, CHD4, HDAC1 and SMAD3 (Figure S7C-S7G). Direct interaction was also observed between BLM and SMAD3 as well as BLM and HDAC1 (Figure S7H). Next, the inventors wanted to determine whether BLM is co-recruited with members of the NuRD and Sin3b complexes onto the CDX2 promoter. Using ChIP, the inventors found that the core ATPase subunits of the two co-repressor complexes, Sin3b and CHD4, are recruited to different binding sites on the CDX2 promoter. While Sin3b was recruited to the SMAD3 binding sites (FIG. 6F), CHD4 is recruited to one of the AP2β sites (FIG. 6G). The extent of recruitment of both Sin3b and CHD4 was always enhanced in cells which express BLM. HDAC1 and HDAC2 are the two common factors present in both NuRD and Sin3b complexes. The inventors found that while HDAC1 was recruited exclusively to SMAD3 binding sites (FIG. 6H), HDAC2 was recruited to the regions where MAD, AP3, SMAD3 and AP2β binding sites were present (FIG. 6I) in a BLM dependent manner. Sequential re-ChIP experiments were carried out which validated that BLM-Sin3b and BLM-CHD4 were binding to specific DNA recognition sequences on the CDX2 promoter (Figure S7I, S7J). Based on these results we hypothesized that BLM repressed CDX2 expression using both Sin3b and NuRD repressor complexes. To obtain direct validation, the inventors carried out ablation experiments using multiple components of the two co-repressor complexes. Hence depletion of Sin3b (FIG. 6J, 6K), CHD4 (FIG. 6L, 6M), HDAC1 (FIG. 6N) and HDAC2 (FIG. 6O) enhanced the expression of CDX2.

Next, the inventors wanted to determine whether in normal colonic tissues BLM indeed was recruited to the CDX2 promoter and thereby negatively regulated the expression of CDX2. To test this hypothesis, the inventors carried out BLM ChIP in eight paired tissue samples from an Indian cohort. The inventors found that the recruitment of BLM to the CDX2 promoter was decreased in the cancerous regions compared to the adjacent normal controls (FIG. 6P), thereby indicating that BLM repressed CDX2 expression in normal colonic tissues compared to the adjacent cancerous region.

### Example 6: Levels of DDSMs are Increased in Colon Cancer Patients

Based on the above experiments the inventors wanted to determine whether the levels of this miR signature is upregulated in colon cancer patient samples. The inventors first analyzed the levels of the DDSMs in the colon cancer patient data in the TCGA database. The inventors found that the levels of six DDSMs (miR-29a-5p, miR-29b-3p, miR-96-5p, miR-182-5p, miR-183-5p, miR-335-3p) were significantly upregulated in both the tissues and blood samples of the colon cancer patients across all the four stages of cancer progression (FIG. 7A, 7B). Next, the inventors expanded their studies to 40 paired tissue samples obtained from the Indian cohort. The levels of the same six miRs were upregulated in the patient cancerous tissue samples compared to their matched adjacent normal controls across stages of cancer progression (FIG. 7C). Compared to the healthy normal individuals, the levels of five of the six circulatory DDSMs were significantly upregulated in the Stage I+II colon cancer patients (FIG. 7D). Kaplan-Meier analysis indicated lesser overall survival for patients with higher risk score (i.e. patients which show higher levels of the combined expression of the six miRs) in both tissue and blood samples (FIG. 7E, 7F).

Further, the inventors wanted to determine whether the increased levels of the DDSMs in the colon cancer patients corelated with the changes in the expression levels of their common target, BRCA1. The inventors found that the transcript levels of BRCA1 decreased in both the colon cancer patient tissue samples and blood compared to their respective matched controls (Figure S8A, S8B). Using Western analysis (Figure S8C, S8D) and immunohistochemistry (Figure S8E, S8F), decreased BRCA1 protein levels was observed in cancerous sections compared to their adjacent normal control. Finally using the TCGA dataset we observed that there existed a negative correlation between the overexpression of DDSMs and the ablation of BRCA1 (FIG. 7G), thereby revealing the pathophysiological significance of the existence of these miRs.

### Example 7: Western Blot and Immunoprecipitation

Western blots were carried out with 50-100 μg of the cell lysates generated in M2 lysis buffer (50 mM Tris pH 7.4, 150 mM NaCl, 10% glycerol, 1% Triton X-100, 0.5 mM EDTA, 0.5 mM EGTA, 1×PIC and 1 mM PMSF). Depending on the antibody the blocking was carried out in BSA, skimmed milk or goat serum. The primary antibody incubations were overnight at 4° C. while the secondary antibody was for 1 hr at room temperature.

RNA immunoprecipitation was carried out in Hela pREV and Ago2 cells according to published protocols (Keene, Komisarow et al., 2006). Cells were scraped in PBS and resuspended in polysome lysis buffer [100 mM KCl, 5 mM MgCl2, 10 mM HEPES (pH 7.0), 0.5% N40, 1 mM DTT, 100 units/ml RNase Out, 400 μM VRC, Protease inhibitor cocktail supplemented with RNase inhibitor and protease inhibitors]. The lysates (2 mg) were used to set up RNA immunoprecipitations with either Protein G bound GFP antibody (2 μg/IP) or with anti-Flag beads (4 μl/IP). The immunoprecipitations were for 4 hours on an end to end rotor at 4° C., after which beads were pelleted at 1200 rpm for 5 minutes and washed with ice cold NT2 buffer 3-4 times. Beads were resuspended in 100 μl of NT2 buffer [50 mm Tris-HCl (pH 7.4), 150 mM NaCl, 1 mM MgCl₂, 0.05% NP40] supplemented with 30 μg of Proteinase K to release the RNP components. This mixture was incubated at 55° C. for 30 minutes. Thereafter, 200 μl of Trizol was added directly to the beads and RNA was isolated. It was followed by cDNA synthesis and qPCR to determine the enrichment of miRNA bound with Argonaute 2 protein. The main steps of traditional immunoprecipitations were similar to the above, except 1×PBS+0.1% NP40 was used as the buffer to make up the volume and carry out washes. Post-IP, the bound proteins were run in SDS-PAGE gels to determine their co-immunoprecipitating partners.

### Example 8: Overexpression and Ablation Studies

GM03509 GFP BLM 4.3.4 or GM03509 GFP Clone 100 cells were transfected with 20 nM of either miRNA inhibitors or miRNA mimics specific to the respective miRs. Lipofectamine 2000 was used for the transfections in 1:1 ratio with the amount of inhibitor or mimic being used. Control miRNA inhibitor or control miRNA mimic at the same concentrations was always used in parallel. Transfections were for 6 hours (hrs). 36 hrs post-transfection either RNA was isolated or lysate was prepared using RIPA buffer [1 mM Tris HCl pH 7.8, 150 mM NaCl, 2% Triton X-100, 1% (w/v) Sodium deoxycholate, 0.1% (w/v) SDS) supplemented with 1×PIC and 1 mM PMSF]. For DNA damage-dependent experiments cells were exposed to 1 mM HU or a gradient of HU concentrations for 16 hrs. Post-exposure, HU was washed off and cells were allowed to grow for 12 hours and this period was called post wash (PW). Cells were also exposed to a particular IR (3Gy) or a range of IR. Lysates were made or RNA extracted +HU, PW or after 1 hr or 6 hrs post-IR exposure. Transfections involving plasmids were carried out using 2-3 μg of the respective plasmids in 6-well cluster plates for 48 hrs. All siRNA transfections were carried out using 200 pmole of the respective siRNAs for 60 hrs. For shSin3b induction, cells transfected with pTRIPZ shSin3b were treated with doxycycline (1 μg/ml) for 48hrs. Corresponding siRNA or shRNA controls were always used in parallel for all the ablation experiments.

### Example 9: Protein Purification

GST tagged proteins were induced in BL-21 cells when the OD reaches 0.4 for 2-3 hrs with 1 mM IPTG at 18° C. for 4 hrs. After induction culture was centrifuged at 6000 rpm for 10 min at 4° C. Pellet was resuspended in 10 ml of GST buffer (50 mM Tris-Cl pH 7.5, 100 mM KCl, 10 mM MgCl₂, 5% glycerol, 0.5% NP-40). Resuspended pellet was sonicated for 4 cycles of 30 sec pulse on and sec off. After sonication whole pellet was centrifuged at 9000 rpm for 30 min. Supernatant and pellet was run on 10% SDS gel to confirm the presence of the respective GST tagged proteins in soluble form. The binding of protein was done with equilibrated GST beads. For this purpose, beads were washed 3 times with GST buffer at 1200 rpm for 5 min at 4° C. After the last wash beads were mixed with equal volume of GST buffer to obtain 50% slurry. Washed beads were incubated with supernatant obtained post-sonication for 4 hrs in an end-to-end rotor. The beads were subsequently washed with GST buffer 3-4 times at 1200 rpm 5 min. The bound proteins were checked by running 10% SDS PAGE. Elution of proteins were done using Poly-Prep Chromatography columns using 5 mM GSH. The fractions with good concentrations and purity of the proteins were pooled, dialyzed in dialysis buffer (25 mM Tris-Cl pH=7.5, 140 mM NaCl, 20% glycerol and 1 mM DTT) for 3 hrs at 4° C., concentration determined and stored in −70° C.

### Example 10: Estimation of mRNA and miRNA Transcripts

RNA was isolated from cells using Trizol reagent according to manufacturer's protocol. For patient samples and animal tissues 50-100 mg of colon adenocarcinoma tissue and adjacent normal tissue samples were crushed in pestle and mortar using liquid nitrogen. The pellet was dried and dissolved in DEPC treated water. cDNA was prepared for the estimation of mRNA levels using Eurogentec-Reverse Transcription Core Kit according to manufacturer's protocol. cDNA synthesis for the miRs was done using mercury LNA™ Universal RT miRNA PCR kit or miRCURY LNA® Kit was used according to manufacturers' protocol. The ct values obtained for the miRs were normalized with U6SnRNA and the ct values obtained for the mRNAs were normalized with GAPDH to get dct value. The dct values were used to obtain relative quantification values which was used to plot the graphs using Prism GraphPad software. Absolute quantitation was carried out to determine the miR levels in patient samples. For this purpose, the dct values were obtained by normalizing the ct values of miRs with U6SnRNA. ddct was calculated by subtracting the dct value of control that is adjacent normal from the dct value of patient that is cancerous tissue. The ddct values were used to calculate the fold change using the formula 2\hat{ }-ddct.

### Example 11: Immunofluorescence and Confocal Analysis

All immunofluorescence experiments were carried out on cells which have been fixed with freshly prepared 4% paraformaldehyde (PFA) for 30 minutes with mild shaking at room temperature. Post-fixation cells were washed with 1×PBS, permeabilized with 1×PBS containing 0.1% Triton-X-100 for 5 minutes with constant shaking at room temperature and fixed with 10% normal chicken serum (NCS) or 1% BSA overnight at 4° C. based on the specific primary antibody. Incubations with both the primary and secondary antibodies were for 1 hour at 37° C. Post washing cells were mounted, with mounting medium containing DAPI (Vector Laboratories). Cells were visualized at magnification followed by imaging using a 63×/1.4 oil immersion objective in a confocal microscope (LSM 510 Meta, Zeiss). The laser lines used were Argon 458/477/488/514 nm (For FITC) and DPSS 561 nm (for Texas Red).

### Example 12: Immunohistochemistry

The poly L lysine coated slides were dipped in 100% xylene for 5 minutes. Subsequently, the slides were dipped in a gradient of isopropanol i.e. 100%, 90%, 70%, 50% for 5 minutes each after which a 3% hydrogen peroxide treatment was given for 30 minutes. The slides were then dipped in EDTA solution (0.05M) and heated at 96° C. for 10 minutes for antigen retrieval. Thereafter, the slides were kept in blocking solution (5% BSA in 1×PBS) for 30 minutes, followed by incubation with the primary antibodies for 1 hr at room temperature. Washing was done in PBST for 5 minutes twice, followed by incubation with secondary antibody HRP Polymer. Washing was done with PBST for 5 minutes twice. The slides were incubated with DAB solution for 2-3 minutes, rinsed with water, counterstained with hematoxylin for 5 mins, washed under running tap water. Sides were then dipped in a gradient of isopropanol, dried and mounted with DPX mounting media.

### Example 13: Comet Assay

Comet assays were carried out according to published protocols (Olive & Banath, 2006). Cells were plated in six well plate, co-transfected with 20 nM of miR control or the indicated miR inhibitors. 48 hours post-transfection, cells were trypsinized and counted. 20,000 cells were resuspended in low melting agarose and a layer of this was coated on a glass slide. Agarose was allowed to gel for 2 minutes and thereafter glass slides coated with agarose were kept in lysis buffer overnight at 37° C. Subsequently slides were submerged in N2 solution [90 mM Tris buffer, 90 mM boric acid, 2 mM Na₂EDTA (pH 8.5)] in an electrophoresis chamber and electrophoresis was conducted at 0.6 Volt/cm. Slides were taken out from the electrophoresis chamber and neutralized by rinsing in autoclaved water. 100 μl of 10 μg/ml Propidium Iodide was put on the agarose for staining Staining was carried out for 20 minutes followed by rinsing in water. Slides were kept in a moistened condition at 4° C. before imaging was carried out in an upright epifluorescence microscope (Carl Zeiss, Germany) at 20×.

### Example 14: Luciferase and Beta-Galactosidase Assays

HEK293T cells, plated in six well cluster, were transfected with the miR promoter constructs cloned in pGL3 basic vector along with full length CDX2 or mini CDX2 construct and CMV β-gal. Lysates were prepared 48 hrs post transfection using luciferase lysis buffer (125 mM Tris-Phosphate pH 7.8, 10 mM EDTA, 5M DTT, 50% glycerol, 5% Triton-X 100). Luciferase assays using equal amount of the lysate were carried out in a 96 well plate using Varioskan Flash (Thermo Scientific). As transfection control, beta-galactosidase assays were carried out for each of the experimental points in parallel. For this equal amount of the lysates were taken in an ELISA 96 well plate. Assays were carried out using the assay buffer Tampon Z (Na₂HPO₄ (12H₂O), NaH₂PO₄ (H₂O), KCl, MgSO₄(7H₂O) in presence of β-mercaptoethanol and ONPG (4 mg/ml). The plate was incubated for 1 minute at room temperature after which reading were taken in Varioskan Flash at 420 nm.

### Example 15: Electrophoretic Mobility Shift Assay (EMSA)

For each EMSA reaction the radiolabeled substrate (10⁴ cpm) was incubated with 500 ng of recombinant CDX2 protein in a binding buffer (10 mM Tris pH-7.5, 50 mM KCl, 2.5 mM MgCl₂, DTT and 4% glycerol) for 15 min at 4° C. 1 μg/μl of poly dI-dC was added in the reaction mixture to prevent non-specific binding. When “supershift” was desired, anti-CDX2 antibody (1 μg/reaction) was added and incubation continued at 37° C. for another 15 min. In certain reactions, 1000× fold-excess cold competitor was also added to confirm the specificity of the assay. Post-completion of the reactions, loading dye (5×TBE, 10% glycerol, 10% bromophenol blue, 1% xylene cyanol, autoclaved H₂O) was added in all the tubes. Samples were then loaded on 6% native PAGE gel which had already been pre-nm in TBE buffer at 20 mA and 300V for 30 min. Sample without CDX2 was used as control for the EMSA reactions. After the run was completed, the gel was dried at 65° C. for 1 hr and exposed overnight, followed by autoradiography.

### Example 16: Sister Chromatid Exchange Analysis

Cells were plated in a six well cluster and transfected with miR inhibitors or miR mimics. 10 mM BrdU was added 3-4 hrs after cells got attached. Cells were allowed to grow for two doubling time period. 20 μl of colcemid was added 2 hrs before completion of doubling time. Thereafter 2 ml of FBS (⅕^{th} diluted in water) was added in each well of the six well cluster and incubated for 30 minutes at 30° C. Cells were fixed using 2 ml fixative A (1:3 acetic acid: methanol v/v) for 15 minutes, after which fixative A was removed and 2 ml fixative B (1:1 fixative A: water v/v) was added and again incubated for 5 minutes. Subsequently fixative B was removed and 2 ml fixative A was again added and incubated for 30 minutes at room temperature. After this incubation, fixative A was removed and another 2 ml of fixative A was again added and incubated for final 15 minutes at 4° C. The coverslips were air dried, followed by addition of 1 ml of 10 μg/ml Hoechst on the coverslips. Cells were kept in dark for 20 minutes, rinsed with 2×SSC (3M sodium chloride, 0.3 M trisodium citrate) buffer and re-incubated in 2×SSC under UV light for 1 hour 45 minutes, after which cells were rinsed with autoclaved water. At this stage, cells were stained with 2 ml Giemsa (4%) for 20 minutes at room temperature. Coverslips were rinsed with water and mounted on slides using DPX mounting medium. All the steps till mounting of the slide was done on six well plate.

### Example 17: Invasion Assay

BD Biocoat Matrigel Invasion chambers were used to assess the invasive property of the cells in vitro, according to the manufacturer's protocol. Warm culture medium was added to the interior of the inserts and bottom of wells, rehydrated for 2 hours in humidified tissue culture incubator, followed by addition of 0.75 ml chemo attractant (media with 10% FBS). Sterile forceps were used to transfer the chambers and control inserts to the wells containing the chemoattractant. The transfected cells (25,000 per well) were resuspended in 0.5 ml of serum free media and seeded in each invasion chamber. In parallel control inserts were also placed. The BD BioCoat Matrigel Invasion Chambers were incubated for 24 hours in a humidified tissue culture incubator, at 37° C., 5% CO2 atmosphere. The invasive cells were able to detach themselves and invaded through the matrigel matrix and the 8μ membrane pores. The membrane was then processed for staining in 1% Toluidiene Blue in 1% Borax (Sigma) and imaging.

### Example 18: Scratch Assay

TG8, TW6 cells were plated in the presence and absence of doxycycline (1 μg/ml in a six well plate. When cells formed a monolayer, a scratch was made with the help of a 2 μl pipette tip. Cells were washed with 1×PBS three times. 2 ml of serum free medium was added to each well. Images were taken after 12, 24, 48, 72 hrs to check the migration of cells. Imaging was done till the gap got filled with cells.

### Example 19: Soft Agar Assay

To study transformation of cells in vitro soft agar assay was carried out. Bottom agar was prepared with 1.6% agarose in water. 1:1 ratio of bottom agar along with 2×DMEM medium was added to each well and allowed to polymerize. Top agar was prepared with 0.8% agarose. TG8/TW6 cells were grown in the presence and absence of doxycycline (1 μg/ml). Cells were trypsinized and counted. 6000 cells were dissolved in 500 μl of bottom agar and 500 μl of 2×DMEM and poured on the top of bottom agar. Plates were kept at 37° C. in the CO₂ incubator. 500 μl of 2×DMEM medium were added on the top when bottom agar started drying. After 15 days, cells were stained with 0.5% crystal violet for 20 minutes. Number of colonies were counted under the microscope.

### Example 20: Small RNA Sequencing

Total RNA was isolated from asynchronously growing BLM isogenic pair of cells—(a) GM03509 GFP-BLM 4.3.4/GM03509 GFP (b) Clone 100 and GM08505 GFP-BLM/GM08505 GFP. RNA was extracted using Trizol and the isolated RNA was used for library preparation using Illumina Small RNA sample preparation kit v1.5 according to the manufacturer's instructions. The total RNA (700-800 ng) were ligated to 3′ and 5′RNA adapters. The ligation products were reverse transcribed using Superscript II Reverse Transcriptase and amplified with 12 cycles of PCR. The PCR products constituting the small RNA cDNA libraries were resolved on 6% Novex TBE PAGE Gel and ˜150 bp fragments excised. The library was eluted from the PAGE gel and analyzed on Agilent 2100 Bioanalyzer using DNA high sensitivity kit (Agilent Technologies, USA). Sequencing of miRNA libraries (˜150 bp fragments) were performed using Illumina GAIIX sequencing platform for 36 cycles. CLC genomic software was used to determine quantitatively the levels of the differentially miRNAa in the two isogenic pairs. With the help of this software, adaptor trimming was also done. The remaining sequence was mapped with the known miRNAs in miR Base database. Subsequently, the mapped miRNAs were normalized to get TPM (Transcript per million). Further analysis was carried out to determine the common miRNAs whose expression was either increased or decreased by the presence of BLM. Only those miRNAs which showed a change above or below two-fold and a p-value either equal to or below 0.05 were chosen for further analysis.

### Example 21: Generation of DNA Binding Mutants of CDX2

Multiple sequence alignment of CDX2 consensus homeobox sequence was carried out with the other homeobox containing proteins to identify all the conserved amino acids. These conserved residues were then identified in other homeodomain proteins which lack DNA binding (Chi, 2005). The subset of the residues were then in silico analyzed using cBio Cancer Genomics Portal (Gao, Aksoy et al., 2013) to identify and discard the hotspots mutations for CDX2 in all forms of cancers. Finally, three amino acids in CDX2 (R190A, R238A and R242A) were identified which were conserved across homeodomains, were not found in any of the cancers and lack the DNA binding activity. CDX2 mutants for these three amino acids were generated and characterized.

### Example 22: In Silico Prediction of Transcription Factors Binding to miR Promoters

In order to determine the transcription factors involved in the regulation of the miRNAs, initial in silico analysis was carried out using the database called ChIP base (rna.sysu.edu.cn/chipbase/). ChIP base is an integrated resource and platform for decoding transcription factor binding. The region upto 5 kb upstream of the TSS as the promoter region of the miRs. Using the ChIP Base database, a list of all the transcription factors binding to the promoter region for all the upregulated miRNAs in BS patient cell lines was determined.

### Example 23: In Silico Prediction and Analysis of miR Targets

Putative targets for each of the miRs were determined by obtaining the results from MiRanda tool. The potential gene targets for each of the miRNAs were predicted were plotted using jvenn software (Bardou, Mariette et al., 2014). Pathway analysis was carried out using Reactome, KEGG and GSEA online databases and the significant pathways with p-value<0.05 were selected.

### Example 24: ChIP and re-ChIP

Cells were plated in 15 cm plates. When the cells reached a confluency of around 90-95%, they were cross-linked using 37% formaldehyde at 25° C. for 20 mins. Cells were scraped in PBS (2 ml) and resuspended in 1 ml of nuclear lysis buffer (1% SDS, 10 mM EDTA, 50 mM Tris-HC1 pH-8.1, 1×PIC). Sonication was done for 38 cycles in a Diagenode Bioruptor keeping maximum amplitude, 30 seconds pulse and 30 seconds hold. Chromatin shearing (300-500 bp) was checked on 1% agarose gel, after which for each ChIP 200 μg of chromatin was incubated with 1 μg of the primary antibody or the corresponding IgG overnight at 4° C. Next day 100 μl of Protein A/G Sepharose beads were added per ChIP reaction and incubated for an additional 2 hours at 4° C. in an end to end rocker. Post-incubation the beads were washed twice with 1 ml of dialysis buffer (0.1% SDS, 1% Triton-X 100, 2 mM EDTA, 20 mM Tris-HCl pH 8.1, 150 mM NaCl, 1×PIC). Subsequently the five washes were done with 1 ml of wash buffer (0.25 M LiCl, 1% sodium deoxycholate, 10 mM Tris-HCl pH 8.1, 1×PIC) followed by the final two washes with 1 ml of TE buffer (10 mM Tris-HCl pH 8.1, 1 mM EDTA). The final pellet was dissolved in 200 μl of TE buffer along with the input samples. Subsequent steps were carried out on both the ChIP and input samples. RNase treatment was carried out by adding RNaseA (100 μg/ml) to the samples, followed by incubation for 30 min at 37° C. Samples were then subjected to reverse cross-linking by adding 4 μl of 10% SDS (final concentration 0.2%), followed by incubating the samples at 70° C. overnight in a thermomixer. Next day Proteinase-K (final concentration 200 μg/ml) was added to each of the samples which were then incubated for 2 hours at 45° C. for Proteinase-K digestion. Phenol-chloroform extractions were performed and finally sample were kept for DNA precipitation overnight at −20° C. Next day samples were centrifuged at 12000 rpm for 90 minutes and the pellet was subjected to a 70% ethanol wash. The DNA pellets were kept for air drying, after which they were dissolved in 20 μl of 10 mM Tris buffer (pH 8.0). DNA estimation was done (by Qubit) and 1 ng of ChIP DNA was used for each ChIP-qPCR reaction. For Re-ChIP, BLM was used as the first antibody while either CHD4 or Sin3b was used as the secondary antibody (2 μg antibody was used in all cases). In this case, the final pellet was dissolved in 200 μl of TE buffer along with 10 mM DTT and incubated at 37° C. for 30 minutes to separate the immunocomplex from protein A/G Sepharose beads. Thereafter, the samples were centrifuged at 1400 rpm for 5 minutes and supernatant containing the immunocomplex was collected in separate eppendorfs. At this stage the second antibody was added to the immunocomplex and incubated overnight at 4° C. in an end to end rocker. Subsequent steps were similar to the normal ChIP protocol. For ChIP on patient samples and their adjacent normal controls, 50-100 mg of the respective tissues were crushed with the help of homogenizer. The rest of the steps were same as that used for cells. The analysis of all the ChIP-qPCR reactions was done using the fold enrichment method. For ChIP involving patient samples, matched pairs of adjacent normal and cancer tissues were chosen.

### Example 25: Nanoparticle Mediated miRNA Delivery

The cationic polymer (TAC6) was used for the in vivo delivery of miRNA inhibitors (Yavvari, Verma et al., 2019). miRNA inhibitors were mixed with TAC6 polymer (final volume 100 μL at 1 mg/mL) and incubated for 20 min at room temperature. Complexes were then coated on incubation with sodium aspartate (final volume 10 μL at 1 mg/mL) for 10 min. Each mouse was given a dose of 200 ng miRNA (50 μL of nanogels) after dilution with PBS and a total of four doses were used.

### Example 26: Mass Spectrometry

The BLM immunoprecipitate was electrophoresed on SDS-PAGE. Coomassie staining was performed. Each lane of the gel containing BLM-interacting proteins was subjected to mass spectrometry. Briefly, each lane was cut out from the gel, separately into smaller pieces. Coomassie stained gel pieces were de-stained using 25 mM ammonium bicarbonate and 50% (v/v) Acetonitrile solution. Subsequently, they were treated with 0.1M TCEP for 45 min at 37° C., followed by 0.5M Iodoacetamide for 1 hr at 37° C. Overnight tryptic in-gel digestion was then carried out, trypsin: protein ratio of 1:100. The next day, peptides were recovered. pH of the supernatant was set to acidic pH (pH˜3) using trifluoroacetic acid. The supernatant was dried in a Speed Vac. Resuspension was carried out in 5% Acetonitrile, 0.1% Formic acid. Desalting was carried out using ZIP TIP (C18 P-10, Millipore). The eluted peptides were dried using speed vac. The peptides were finally resuspended in 5% Acetonitrile, 0.1% Formic acid) and were subjected to LC MS/MS analysis using EASY-nLC system (Thermo Fisher Scientific). The Sin3b and CHD4 peptides were obtained three times (biological replicates).

### Example 27: Animal Studies

All animal studies were carried out in National Institute of Immunology according to an approved animal ethics protocol (IAEC approval reference number: IAEC #398/15). Following animal studies were carried out: (A) Tumorigenic potential of HCT116 BLM−/− cells expressing miR inhibitors using a subcutaneous model; (B) Tumorigenic potential of HCT116 BLM−/− cells which were challenged with a nanoparticle coated miR inhibitors in a subcutaneous model; (C) Tumorigenic potential of HC1/HW2 cells using a subcutaneous model; (D) Tumorigenic potential of TG8/TW6 cells using a subcutaneous model; (E) Tumorigenic potential of TG8/TW6 cells using an intravenous model; (F) Tumorigenic potential of TG8/TW6 cells using an orthotopic model. In all subcutaneous models (A-D) approximately 2 million cells were resuspended in Fetal Bovine Serum. These cells were injected subcutaneously in NOD SCID mice along with matrigel. In all the TG8/TW6 models (D-F) the mice were additionally treated with doxycycline (10 mg/ml/kg body weight) by oral gavage every day throughout the experiments. In all the subcutaneous models, tumor formation started after 7-10 days. In case of (B) a nanoparticle mediated delivery system was used to deliver miR inhibitor Control, miR inhibitor 29a-5p or miR inhibitor 96-5p directly to the base of the tumors every third day for 4 times. In case of intravenous model (E) or orthotopic model (F), 50,000 TG8/TW6 cells were used. Cells were either injected via tail vain (E) or were implanted into the cecal wall (F). At the indicated end points, whole body imaging was done for the mice (in D-F) using a in vivo imaging system (Perkin Elmer) to check the expression of GFP and thereby determine the invasive potential of TG8/TW6 cells. For all the above models, at the end point the mice were sacrificed by cervical dislocation. The excised tumors (in case of subcutaneous models, A-D) were imaged, measured, used for lysate preparation (using RIPA), RNA extraction (using Trizol LS) and immunohistochemistry.

### Example 28: Patient Sample miRNA Analysis

### 28.1: TCGA Analysis

miRNA expression levels of 332 colon tumor samples, 82 blood samples and 8 normal samples were downloaded from the GDC data portal (TCGA COAD) using the miR quantitation file and files with extension FPKM.txt.gz, respectively on 27 Apr. 2017. The clinical information for all these samples were also downloaded from the GDC data portal. The log₂(x+1) transformation was carried out on the miR levels and gene expression values. The expression of these miRs in different stages of the colon cancer as compared to the normal samples was then examined by comparing the means of the expression values across different stages. Kruskal-Wallis test was carried out in SPSS v.24 to compare the means of the miR expression across stages with that in the normal samples. The number of patients (n) in each classification is as follows: Normal: 8, Stage I: 67, Stage II: 166, Stage III: 119, Stage IV: 61.

For Km analysis, the expression value in each tissue sample and blood sample was subtracted from the average value of normal samples. These values were then used to calculate the risk score of the 6 miRs significantly found upregulated in TCGA samples as described earlier (Ji, Qiao et al., 2018). Briefly, regression coefficients (β) of the individual miRNA were determined by Cox regression analysis. The risk score was calculated for each patient using the formula: (β_{miR29a-5 p*} expression value of miR-29a-5p)+(β_{miR29a-3p*} expression value of miR-29b-3p)+(β_{miR96-5p*} expression value of miR-96-5p)+(β_{miR182-5p*} expression value of miR-182-5p)+(β_{miR183-5p*} expression value of miR-183-5p)+(β_{miR335-5p*} expression value of miR-335-3p). The tissue samples with risk score more than or equal to 75% quartile or 0.9618 were grouped as high-risk samples (n=78) while the patients with risk score less than or equal to 25% quartile or −0.3654 were grouped as low-risk samples (n=76). Similarly, for blood samples, high risk group (n=20) had risk score more than or equal to 1.224 while the low risk group (n=20) had score less than or equal to −0.2559. The overall survival (OS) curves were plotted using Kaplan-Meier analysis in SPSS followed by log-rank test to detect the significant difference between the high and low risk group of patients. Spearman correlation was carried out with 207 patient samples for which both the transcriptome and miR expression datasets were available.

### 28.2 Indian Cohort

All patients pertaining to the Indian cohort were obtained from All India Institute of Medical Science (according to Institute Human Ethics approval number RP-23/2017). All experimental work on these samples was carried out in National Institute of Immunology (according to Institute Human Ethics approval number IHEC #92/17). Cancerous and matched adjacent normal tissues were obtained from forty colon cancer patients. The adjacent normal tissues were excised 7-10 cm from the periphery of the tumor. Each tissue sample was graded, subjected to routine histology and H and E staining, based on which the core of the tumour was used for RNA/protein/IHC analysis. Patients in polyp, stages I and II were combined together (n=26) while stages III and IV were combined together (n=14). For miR analysis in blood, the −Log transformed values from thirty-three blood samples from healthy normal individuals were plotted and compared with the blood obtained from the forty Indian colon cancer patients Mann-Whitney test was used to identify the significantly altered expression of miRs, BRCA1 mRNA and BRCA1 protein (estimated by both western and IHC) in the tissues of the Indian cohort. The differential levels of the miRs which were statistically altered in the blood of Indian colon cancer patient was also determined by Mann-Whitney test.

1. Deoxyribo-Nucleic Acid (DNA) damage sensitive microRNA signatures (DDSMs), comprising one or more sequences, wherein the one or more sequences comprise SEQ ID NO. 1, 2, 3, 4, 5, 6, 7, 8, 9, 10, 11, 12, 13, 14, 15, 16, and combinations thereof.

2. The DDSMs of claim 1, wherein said DDSMs are upregulated by a DNA damage inducible transcription factor.

3. The DDSMs of claim 1, wherein said DNA damage inducible transcription factor is CDX2.

4. The DDSMs of claim 1, wherein said DDSMs find application as prognostic and diagnostic biomarkers.

5. The DDSMs of claim 1, wherein said DDSMs are for qualitative and quantitative estimation of specific microRNA levels in different stages in colon cancer patients.

6. The DDSMs of claim 1, wherein the DDSMs are for detection of colon cancer.

7. The DDSMs of claim 1, wherein detection of the DDSMs is performed in at least one sample, wherein the sample is tissue or body fluid.

8. A method of diagnosing tumor growth, comprising detecting the DDSMs of claim 1, wherein said DDSMs are upregulated by CDX2.

9. The method of claim 8, wherein said DDSMs when upregulated decrease the expression of DNA damage protein BRCA1, ATM, RNF8, or Chk1, or combinations thereof.

10. A method for identifying Deoxyribo-Nucleic Acid (DNA) damage sensitive microRNA signatures (DDSMs), which respond to level of DNA damage, wherein said method comprises the steps of:
a. isolating RNA from two pair of isogenic cell lines, one with and one without BLM helicase;
b. conducting small RNA sequencing with isolated RNA of step (a);
c. observing expression levels of micro RNAs (miRNAs, miRs) in both the isogenic pairs in absence of BLM helicase expression;
d. validating relative expression of upregulated miRs obtained from step (b) in the same isogenic pairs of cells;
e. further validating the expression of upregulated miRs in isogenic lines of colon cancer origin; and
f. identifying DDSMs having upregulated by common transcription factor CDX2.

11. The method of claim 10 wherein said isogenic pairs of cells are selected from immortalized cells from GM03509 expressing with GFP-BLM or GFP or immortalized cells from GM08505 expressing with GFP-BLM or GFP.

12. A method of treatment of cancer, comprising administering to a colon cancer patient in need thereof miR inhibitors against Deoxyribo-Nucleic Acid (DNA) damage sensitive microRNA signatures (DDSMs) selected from SEQ ID NO. 1, 2, 3, 4, 5, 6, 7, 8, 9, 10, 11, 12, 13, 14, 15 and 16 into tumours of the colon cancer patient.

13. The method of claim 12, wherein said miR inhibitors are delivered along with nanoparticle or hydrogel or adenoviral based delivery system.

14. A kit for detecting Deoxyribo-Nucleic Acid (DNA) damage sensitive microRNA (miR) signatures (DDSMs) and levels of CDX2 expression, wherein said kit comprises a microfluidic system in which patient's body fluid or tissue is added, from which miR and mRNA are extracted, converted into complementary DNA (cDNA) by reverse transcription PCR (RT-PCR), and a level of the cDNA quantitatively determined.

15. The DDSMs of claim 7, wherein the body fluid is blood, plasma, urine, or sputum.

16. The kit of claim 14, wherein the body fluid is blood, plasma, urine, or sputum.

17. The kit of claim 14, wherein the tissue is colon cancer tissue.

18. The kit of claim 14, wherein the patient is a colon cancer patient or a patient at risk of having colon cancer.


SEQUENCE LISTING (16 sequences)

SEQ ID NO: 1 - RNA, 22 nt, Homo sapiens
acugauuucuuuugguguucag

SEQ ID NO: 2 - RNA, 23 nt, Homo sapiens
uagcaccauuugaaaucaguguu

SEQ ID NO: 3 - RNA, 23 nt, Homo sapiens
uuuggcacuagcacauuuuugcu

SEQ ID NO: 4 - RNA, 23 nt, Homo sapiens
ucuacagugcacgugucuccagu

SEQ ID NO: 5 - RNA, 24 nt, Homo sapiens
uuuggcaaugguagaacucacacu

SEQ ID NO: 6 - RNA, 22 nt, Homo sapiens
uauggcacugguagaauucacu

SEQ ID NO: 7 - RNA, 22 nt, Homo sapiens
uuuuucauuauugcuccugacc

SEQ ID NO: 8 - RNA, 22 nt, Homo sapiens
acuggacuuggagucagaaggc

SEQ ID NO: 9 - RNA, 22 nt, Homo sapiens
uccuguacugagcugccccgag

SEQ ID NO: 10 - RNA, 22 nt, Homo sapiens
aucaaggaucuuaaacuuugcc

SEQ ID NO: 11 - RNA, 22 nt, Homo sapiens
uuaugguuugccugggacugag

SEQ ID NO: 12 - RNA, 21 nt, Homo sapiens
auaguccgaguaacgucgggc

SEQ ID NO: 13 - RNA, 21 nt, Homo sapiens
gacuauagaacuuuccccuca

SEQ ID NO: 14 - RNA, 22 nt, Homo sapiens
ucagguguggaaacugaggcag

SEQ ID NO: 15 - RNA, 23 nt, Homo sapiens
aggaugagcaaagaaaguagauu

SEQ ID NO: 16 - RNA, 21 nt, Homo sapiens
cggggcagcucaguacaggau